(19) United States
(12) Patent Application Publication
(10) Pub. No.: US 2007/0244138 A1
(43) Pub. Date: Oct. 18, 2007

(54) Mercaptoimidazoles as Ccr2 Receptor Antagonists

(75) Inventors: Gustaaf Maria Boeckx, Oud-Turnhout (BE); Guy Rosalia Eugeen Van Lommen, Berlaar (BE); Julien Georges Pierre-Olivier Doyon, Turnhout (BE); Erwin Coesemans, Nijlen (BE)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: JANSSEN PHARMACEUTICA N.V., Beerse (BE)

(21) Appl. No.: 11/569,321

(22) PCT Filed: May 24, 2005
(86) PCT No.: PCT/EP05/52373

§ 371 Date: Nov. 17, 2006

(30) Foreign Application Priority Data
May 26, 2004 (EP) PCT/EP04/50935

(51) Int. Cl. A61K 31/41; A61K 31/505; A61P 29/00; C07D 233/00; C07D 239/02; C07D 257/04; C07D 271/06; C07D 271/10

(52) U.S. Cl. 514/275; 514/364; 514/381; 514/385; 544/297; 548/131; 548/143; 548/250; 548/316.4

(57) ABSTRACT

The present invention relates to a compound of formula (I) 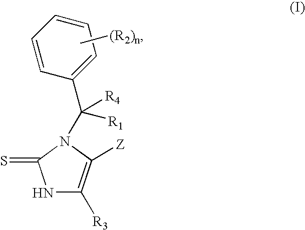
a N-oxide, a pharmaceutically acceptable addition salt, a quaternary amine or a stereochemically isomeric form thereof, wherein R₁ represents hydrogen, C₁₋₆alkyl, C₃₋₇cycloalkyl, C₁₋₆alkyloxyC₁₋₆alkyl, di(C₁₋₆alkyl)aminoC₁₋₆alkyl, aryl or heteroaryl; each R₂ independently represents halo, C₁₋₆alkyl, C₁₋₆alkyloxy, C₁₋₆alkylthio, polyhaloC₁₋₆alkyl, polyhaloC₁₋₆alkyloxy, cyano, aminocarbonyl, amino, mono- or di(C₁₋₄alkyl) amino, nitro, aryl or aryloxy; R₃ represents hydrogen, cyano, optionally substituted C₁₋₆alkyl, C(═O)—O—R₅, C(═O)—NR₆ₐR_{6b}, C(═S)—NR₆ₐR_{6b}, S(═O)₂—NR₆ₐR_{6b} or C(═O)—R₇; R₄ represents hydrogen or C₁₋₆alkyl; n is 1, 2, 3, 4 or 5; Z represents a cyclic ring system. The invention also relates to processes for preparing the compounds of formula (I), their use as CCR2 antagonists and pharmaceutical compositions comprising them.

The present invention concerns mercaptoimidazole derivatives having CCR2 receptor antagonistic properties. The invention further relates to methods for their preparation and pharmaceutical compositions comprising them. The invention also relates to the use of said compounds for the manufacture of a medicament for the prevention or the treatment of diseases mediated through activation of the CCR2 receptor, in particular the CCR2B receptor.

WO 02/066458 describes 2-thio-substituted imidazole derivatives having immunomodulating and/or inhibiting activity on the release of cytokines, especially TNF-α and IL-β.

FR 1,487,326 relates to thio-imidazole derivatives useful as analgetic and for its vasodilatation activity.

FR 6,751 M describes thio-imidazole derivatives as sedatives and analgesics. U.S. Pat. No. 3,850,944 describes 2-mercapto-5-(3-pyridyl)-imidazole derivatives having antiinflammatory activity.

EP 0,277,384 describes 1H-imidazole-5-carboxylic acid derivatives for controlling weeds.

The compounds of the invention differ from the prior art compounds in structure, in their pharmacological activity and/or pharmacological potency.

One aspect of the present invention relates to a compound of formula 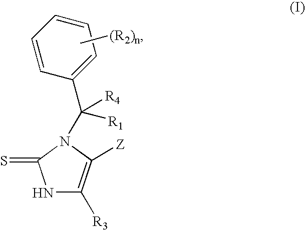
a N-oxide, a pharmaceutically acceptable addition salt, a quaternary amine or a stereochemically isomeric form thereof, wherein
R₁ represents hydrogen, C₁₋₆alkyl, C₃₋₇cycloalkyl, C₁₋₆alkyloxyC₁₋₆alkyl, di(C₁₋₆alkyl)aminoC₁₋₆alkyl, aryl or heteroaryl;
each R₂ independently represents halo, C₁₋₆alkyl, C₁₋₆alyloxy, C₁₋₆alkylthio, polyhaloC₁₋₆alkyl, polyhaloC₁₋₆alkyloxy, cyano, aminocarbonyl, amino, mono- or di(C₁₋₄alkyl)amino, nitro, aryl or aryloxy;
R₃ represents hydrogen, cyano, C₁₋₆alkyl optionally substituted with hydroxy or C₁₋₆alkyloxy, C(═O)—O—R₅, C(═O)—NR₆ₐR_{6b}, C(═S)—NR₆ₐR_{6b}, S(═O)₂—NR₆ₐR_{6b} or C(═O)—R₇;
R₄ represents hydrogen or C₁₋₆alkyl;
R₅ represents hydrogen, C₁₋₆alkyl, hydroxyC₁₋₆alkyl, C₂₋₆alkenyl, C₂₋₆alkynyl, polyhaloC₁₋₆alkyl, C₁₋₆alkyloxyC₁₋₆alkyl, aminoC₁₋₆alkyl, mono- or di(C₁₋₄alkyl)aminoC₁₋₆alkyl aminocarbonylC₁₋₆alkyl mono- or di(C₁₋₄alkyl)aminocarbonylC₁₋₆alkyl or aryl;
R₆ₐ and R_{6b} each independently represent hydrogen, C₁₋₆alkyl, amino, mono- or di(C₁₋₄alkyl)amino, arylNH—, aminoC₁₋₆alkyl, mono- or di(C₁₋₄alkyl)amino-C₁₋₆alkyl, C₁₋₆alkylcarbonylamino, aminocarbonylamino, C₁₋₆alkyloxy, carbonylamino or hydroxyC₁₋₆alkyl; or
R₆ₐ and R_{6b} taken together with the nitrogen to which they are attached form pyrrolidinyl, imidazolidinyl, pyrazolidinyl, piperidinyl, piperazinyl, morpholinyl, thiomorpholinyl or piperazinyl substituted with C₁₋₆alkyl;
R₇ represents hydrogen, C₁₋₆alkyl, hydroxyC₁₋₆alkyl, C₂₋₆alkenyl, C₂₋₆alkynyl, polyhaloC₁₋₆alkyl, C₁₋₆alkyloxy, C₁₋₆alkyl, aminoC₁₋₆alkyl, mono- or di(C₁₋₄alkyl)aminoC₁₋₆alkyl, aminocarbonylC₁₋₆alkyl, mono- or di(C₁₋₄alkyl)aminocarbonylC₁₋₆alkyl, aryl or heteroaryl;
Z represents a cyclic ring system selected from 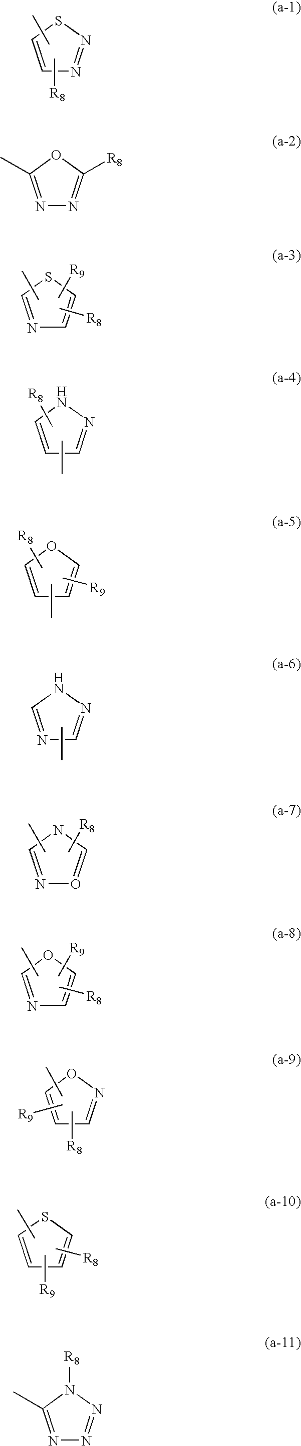 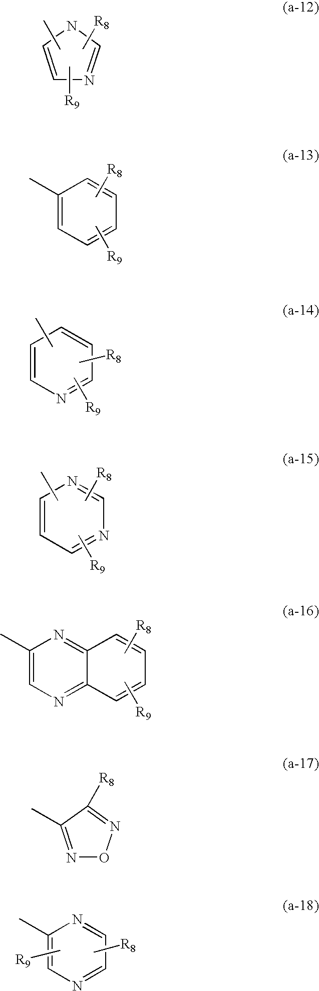
each R₈ independently represents hydrogen, halo, C₁₋₆alkyl, C₁₋₆alkyloxy, polyhaloC₁₋₆alkyl, polyhaloC₁₋₆alkyloxy, cyano, aminocarbonyl, mono- or di(C₁₋₄alkyl)aminocarbonyl, amino, mono- or di(C₁₋₄alkyl)amino, hydroxyC₁₋₆alkylamino, aryl, aryloxy, piperidinyl, piperidinylamino, morpholinyl, piperazinyl or nitro;
each R₉ independently represents hydrogen, halo or C₁₋₆alkyl;
n is 1, 2, 3, 4 or 5;
aryl represents phenyl or phenyl substituted with one, two, three, four or five substituents each independently selected from halo, C₁₋₆alkyl, C₁₋₆alkyloxy, polyhaloC₁₋₆alkyl, polyhaloC₁₋₆alkyloxy, cyano, aminocarbonyl, mono- or di(C₁₋₄alkyl)aminocarbonyl, amino, mono- or di(C₁₋₄alkyl)amino, phenyloxy or nitro;
heteroaryl represents furanyl, thienyl, pyrrolyl, oxazolyl, thiazolyl, imidazolyl, pyrazolyl, isoxazolyl, isothiazolyl, oxadiazolyl, triazolyl, thiadiazolyl, pyridyl, pyridazinyl, pyrimidinyl, pyrazinyl, each of said heterocycles optionally being substituted with one or two substituents each independently selected from halo, C₁₋₆alkyl, C₁₋₆alkyloxy, polyhaloC₁₋₆alkyl, polyhaloC₁₋₆alkyloxy, cyano, aminocarbonyl, mono- or di(C₁₋₄alkyl)aminocarbonyl, amino, mono- or di(C₁₋₄alkyl)amino or nitro.

The present invention also relates to the use of a compound for the manufacture of a medicament for preventing or treating diseases mediated through activation of the CCR2 receptor, in particular for preventing or treating inflammatory diseases, wherein said compound is a compound of formula (I) 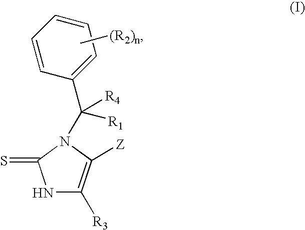
a N-oxide, a pharmaceutically acceptable addition salt, a quaternary amine or a stereochemically isomeric form thereof, wherein
R₁ represents hydrogen, C₁₋₆alkyl, C₃₋₇cycloalkyl, C₁₋₆alkyloxyC₁₋₆alkyl, di(C₁₋₆alkyl)aminoC₁₋₆alkyl, aryl or heteroaryl;
each R₂ independently represents halo, C₁₋₆alkyl, C₁₋₆alkyloxy, C₁₋₆alkylthio, polyhaloC₁₋₆alkyl, polyhaloC₁₋₆alkyloxy, cyano, aminocarbonyl, amino, mono- or di(C₁₋₄alkyl)amino, nitro, aryl or aryloxy;
R₃ represents hydrogen, cyano, C₁₋₆alkyl optionally substituted with hydroxy or C₁₋₆alkyloxy, C(═O)—O—R₅, C(═O)—N₆ₐR_{6b}, C(═S)—NR₆ₐR_{6b}, S(═O)₂—NR₆ₐR_{6b} or C(═O)—R₇;
R₄ represents hydrogen or C₁₋₆alkyl;
R₅ represents hydrogen, C₁₋₆alkyl, hydroxyC₁₋₆alkyl, C₂₋₆alkenyl, C₂₋₆alkynyl, polyhaloC₁₋₆alkyl, C₁₋₆alkyloxyC₁₋₆alkyl, aminoC₁₋₆akyl, mono- or di(C₁₋₄alkyl)aminoC₁₋₆alkyl, aminocarbonylC₁₋₆alkyl, mono- or di(C₁₋₄alkyl)aminocarbonylC₁₋₆alkyl or aryl;
R₆ₐ and R_{6b} each independently represent hydrogen, C₁₋₆alkyl, amino, mono- or di(C₁₋₄alkyl)amino, arylNH—, aminoC₁₋₆alkyl, mono- or di(C₁₋₄alkyl)amino C₁₋₆alkyl, C₁₋₆alkylcarbonylamino, aminocarbonylamino, C₁₋₆alkyloxy, carbonylamino or hydroxyC₁₋₆alkyl; or
R₆ₐ and R_{6b} taken together with the nitrogen to which they are attached form pyrrolidinyl, imidazolidinyl, pyrazolidinyl, piperidinyl, piperazinyl, morpholinyl, thiomorpholinyl or piperazinyl substituted with C₁₋₆alkyl;
R₇ represents hydrogen, C₁₋₆alkyl, hydroxyC₁₋₆alkyl, C₂₋₆alkenyl, C₂₋₆alkynyl, polyhaloC₁₋₆akyl, C₁₋₆alkyloxyC₁₋₆alkyl, aminoC₁₋₆alkyl, mono- or di(C₁₋₄alkyl)aminoC₁₋₆alkyl, aminocarbonylC₁₋₆alkyl, mono- or di(C₁₋₄alkyl)aminocarbonylC₁₋₆alky, aryl or heteroaryl;
Z represents a cyclic ring system selected from 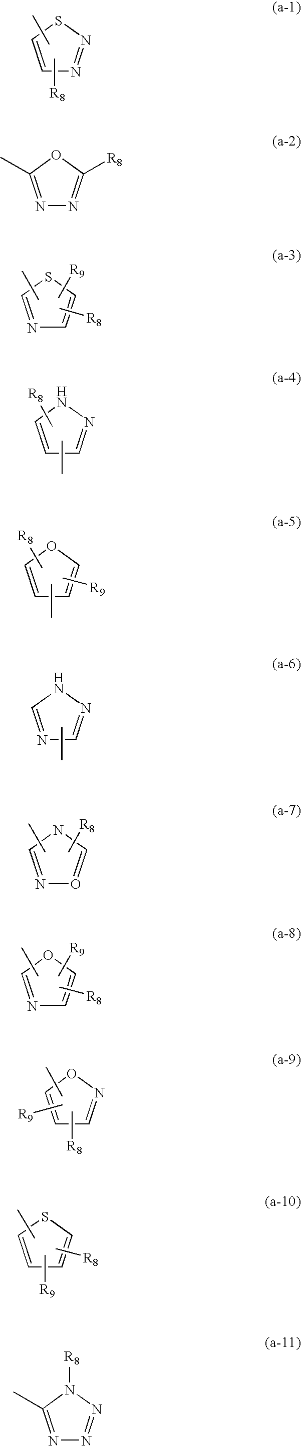 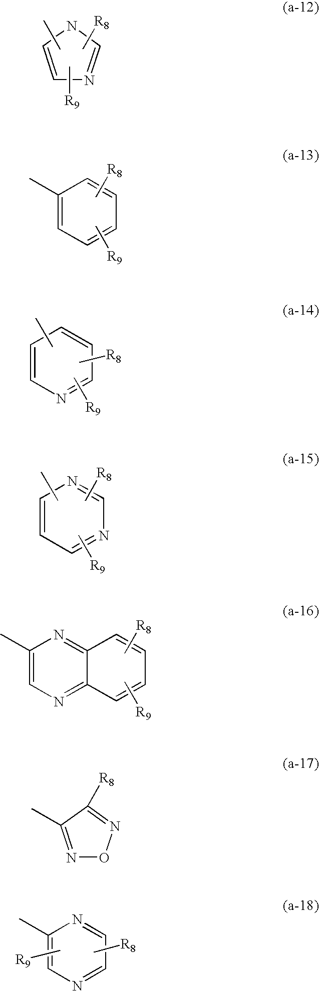
each R independently represents hydrogen, halo, C₁₋₆alkyl, C₁₋₆alkyloxy, polyhaloC₁₋₆alkyl, polyhaloC₁₋₆alkyloxy, cyano, aminocarbonyl, mono- or di(C₁₋₄alkyl)aminocarbonyl, annno, mono- or di(C₁₋₄alkyl)amino, hydroxyC₁₋₆alkylamino, aryl, aryloxy, piperidinyl, piperidinylamino, morpholinyl, piperazinyl or nitro;
each R₉ independently represents hydrogen, halo or C₁₋₆alkyl;
n is 1, 2, 3, 4 or 5;
aryl represents phenyl or phenyl substituted with one, two, three, four or five substituents each independently selected from halo, C₁₋₆alkyl, C₁₋₆alkyloxy, polyhaloC₁₋₆alkyl, polyhaloC₁₋₆alkyloxy, cyano, aminocarbonyl, mono- or di(C₁₋₄alkyl)aminocarbonyl, amino, mono- or di(C₁₋₄alkyl)amino, phenyloxy or nitro
heteroaryl represents furanyl, thienyl, pyrrolyl, oxazolyl, thiazolyl, imidazolyl, pyrazolyl, isoxazolyl, isothiazolyl, oxadiazolyl, triazolyl, thiadiazolyl, pyridyl, pyridazinyl pyrimidinyl, pyrazinyl, each of said heterocycles optionally being substituted with one or two substituents each independently selected from halo, C₁₋₆alkyl, C₁₋₆alkyloxy, polyhaloC₁₋₆alkyl, polyhaloC₁₋₆alkyloxy, cyano, aminocarbonyl, mono- or di(C₁₋₄alkyl)aminocarbonyl, amino, mono- or di(C₁₋₄alkyl)amino or nitro.

As used hereinbefore or hereinafter C₁₋₄alkyl as a group or part of a group defines straight or branched chain saturated hydrocarbon radicals having from 1 to 4 carbon atoms such as methyl, ethyl, propyl, 1-methylethyl, butyl; C₁₋₆alkyl as a group or part of a group defines straight or branched chain saturated hydrocarbon radicals having from 1 to 6 carbon atoms such as the group defined for C₁₋₄alkyl and pentyl, hexyl, 2-methylbutyl and the like; C₃₋₇cycloalkyl is generic to cyclopropyl, cyclobutyl, cyclopentyl, cyclohexyl and cycloheptyl; C₂₋₆alkenyl defines straight and branched chain hydrocarbon radicals having from 2 to 6 carbon atoms containing a double bond such as ethenyl, propenyl, butenyl, pentenyl, hexenyl and the like; C₂₋₆alkynyl defines straight and branched chain hydrocarbon radicals having from 2 to 6 carbon atoms containing a triple bond such as ethynyl, propynyl, butynyl, pentynyl, hexynyl and the like.

As used hereinbefore, the term (═O) forms a carbonyl moiety when attached to a carbon atom, a sulfoxide moiety when attached to a sulfur atom and a sulfonyl moiety when two of said terms are attached to a sulfur atom.

The term halo is generic to fluoro, chloro, bromo and iodo. As used in the foregoing or hereinafter, polyhalomethyl as a group or part of a group is defined as mono- or polyhalosubstituted methyl, in particular methyl with one or more fluoro atoms, for example, difluoromethyl or trifluoromethyl; polyhaloC₁₋₆alkyl as a group or part of a group is defined as mono- or polyhalosubstituted C₁₋₆alkyl, for example, the groups defined in polyhalomethyl, 1,1-difluoro-ethyl and the like. In case more than one halogen atoms are attached to an alkyl group within the definition of polyhalomethyl or polyhaloC₁₋₆alkyl, they may be the same or different.

The term heteroaryl in the definition of R₁ or R₇ is meant to include all the possible isomeric forms of the heterocycles, for instance, pyrrolyl comprises 1H-pyrrolyl and 2H-pyrrolyl.

The aryl, heteroaryl, heterocyclic ring systems or cyclic ring systems listed in the definitions of the substituents of the compounds of formula (I) (see for instance R₁, R₅, R₇ and Z) as mentioned hereinabove or hereinafter may be attached to the remainder of the molecule of formula (I) through any ring carbon or heteroatom as appropriate, if not otherwise specified. Thus, for example, when heteroaryl is imidazolyl, it may be 1-imidazolyl, 2-imidazolyl, 4-imidazolyl and the like.

When any variable (eg. R₆ₐ, R_{6b}) occurs more than one time in any constituent, each definition is independent.

Lines drawn from substituents into ring systems indicate that the bond may be attached to any of the suitable ring atoms. When the lines are drawn into bicyclic ring systems, it indicates that the bond may be attached to any of the suitable ring atoms of any one of the two cycles of the bicyclic ring system.

For therapeutic use, salts of the compounds of formula (I) are those wherein the counterion is pharmaceutically acceptable. However, salts of acids and bases which are non-pharmaceutically acceptable may also find use, for example, in the preparation or purification of a pharmaceutically acceptable compound. All salts, whether pharmaceutically acceptable or not are included within the ambit of the present invention.

The pharmaceutically acceptable addition salts as mentioned hereinabove are meant to comprise the therapeutically active non-toxic acid addition salt forms which the compounds of formula (I) are able to form. The latter can conveniently be obtained by treating the base form with such appropriate acids as inorganic acids, for example, hydrohalic acids, e.g. hydrochloric, hydrobromic and the like; sulfuric acid; nitric acid; phosphoric acid and the like; or organic acids, for example, acetic, propanoic, hydroxy-acetic, 2-hydroxypropanoic, 2-oxopropanoic, oxalic, malonic, succinic, maleic, fumaric, malic, tartaric, 2-hydroxy-1,2,3-propanetricarboxylic, methanesulfonic, ethanesulfonic, benzenesulfonic, 4-methylbenzenesulfonic, cyclohexanesulfamic, 2-hydroxybenzoic, 4-amino-2-hydroxybenzoic and the like acids. Conversely the salt form can be converted by treatment with alkali into the free base form.

The compounds of formula (I) containing acidic protons may be converted into their therapeutically active non-toxic metal or amine addition salt forms by treatment with appropriate organic and inorganic bases. Appropriate base salt forms comprise, for example, the ammonium salts, the alkali and earth alkaline metal salts, e.g. the lithium, sodium, potassium, magnesium calcium salts and the like, salts with organic bases, e.g. primary, secondary and tertiary aliphatic and aromatic amines such as methylamine, ethylamine, propylamine, isopropylamine, the four butylamine isomers, dimethylamine, diethylamine, diethanolamine, dipropylamine, diisopropylamine, di-n-butylanine, pyrrolidine, piperidine, morpholine, trimethylamine, triethylamine, tripropylamine, quinuclidine, pyridine, quinoline and isoquinoline, the benzathine, N-methyl-D-glucamine, 2-amino-2-(hydroxymethyl)-1,3-propanediol, hydrabamine salts, and salts with amino acids such as, for example, arginine, lysine and the like. Conversely the salt form can be converted by treatment with acid into the free acid form.

The term addition salt also comprises the hydrates and solvent addition forms which the compounds of formula (I) are able to form. Examples of such forms are e.g. hydrates, alcoholates and the like.

The term “quaternary amine” as used hereinbefore defines the quaternary ammonium salts which the compounds of formula (I) are able to form by reaction,between a basic nitrogen of a compound of formula (I) and an appropriate quaternizing agent, such as, for example, an optionally substituted alkylhalide, arylhalide or arylalkylhalide, e.g. methyliodide or benzyliodide. Other reactants with good leaving groups may also be used, such as alkyl trifluoromethanesulfonates, alkyl methanesulfonates, and alkyl p-toluenesulfonates. A quaternary amine has a positively charged nitrogen. Pharmaceutically acceptable counterions include chloro, bromo, iodo, trifluoroacetate and acetate. The counterion of choice can be introduced using ion exchange resins.

The N-oxide forms of the present compounds are meant to comprise the compounds of formula (I) wherein one or several tertiary nitrogen atoms are oxidized to the so-called N-oxide.

It will be appreciated that some of the compounds of formula (I) and their N-oxides, addition salts, quaternary amines and stereochemically isomeric forms may contain one or more centers of chirality and exist as stereochemically isomeric forms.

The term “stereochemically isomeric forms” as used hereinbefore defines all the possible stereoisomeric forms which the compounds of formula (I), and their N-oxides, addition salts, quaternary amines or physiologically functional derivatives may possess. Unless otherwise mentioned or indicated, the chemical designation of compounds denotes the mixture of all possible stereochemically isomeric forms, said mixtures containing all diastereomers and enantiomers of the basic molecular structure as well as each of the individual isomeric forms of formula (I) and their N-oxides, salts, solvates or quaternary amines substantially free, i.e. associated with less than 10%, preferably less than 5%, in particular less than 2% and most preferably less than 1% of the other isomers. Thus, when a compound of formula (I) is for instance specified as (E), this means that the compound is substantially free of the (Z) isomer.

In particular, stereogenic centers may have the R- or S-configuration; substituents on bivalent cyclic (partially) saturated radicals may have either the cis- or trans-configuration. Compounds encompassing double bonds can have an E (entgegen) or Z (zusammen)-stereochemistry at said double bond. The terms cis, trans, R, S, E and Z are well known to a person skilled in the art.

Stereochemically isomeric forms of the compounds of formula (I) are obviously intended to be embraced within the scope of this invention.

Some of the compounds of formula (I) may also exist in their tautomeric form. Such forms although not explicitly indicated in the above formula (I) are intended to be included within the scope of the present invention. For instance, it is intended that formula (I) includes the tautomeric form of 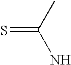
being 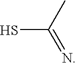
Thus, the compounds of the present invention include compounds of formula

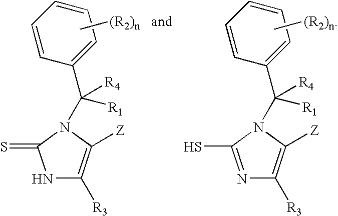

Whenever used hereinafter, the term “compounds of formula (I)” is meant to also include their N-oxide forms, their addition salts, their quaternary amines and their stereochemically isomeric forms. Of special interest are those compounds of formula (I) which are stereochemically pure.

Whenever used hereinbefore or hereinafter that substituents can be selected each independently out of a list of numerous definitions, such as for example for R₆ₐ or R_{6b}, all possible combinations are intended which are chemically possible.

A first interesting embodiment of the present invention relates to a compound of formula 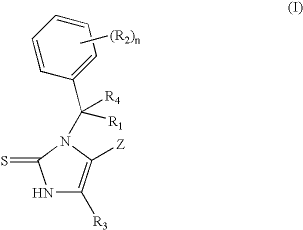
wherein
R₁ represents hydrogen, C₁₋₆alkyl, C₃₋₇cycloalkyl, C₁₋₆alkyloxyC₁₋₆alkyl, di(C₁₋₆alkyl)aminoC₁₋₆alkyl, aryl or heteroaryl;
each R₂ independently represents halo, C₁₋₆alkyl, C₁₋₆alkyloxy, C₁₋₆alkylthio, polyhaloC₁₋₆alkyl, polyhaloC₁₋₆alkyloxy, cyano, aminocarbonyl, amino, mono- or di(C₁₋₄alkyl)amino, nitro, aryl or aryloxy;
R₃ represents hydrogen, cyano, C₁₋₆alkyl optionally substituted with hydroxy or C₁₋₆alkyloxy, C(═O)—O—R₅, C(═O)—NR₆ₐR_{6b}, C(═S)—NR₆ₐR_{6b}, S(═O)₂—NR₆ₐR_{6b} or C(═O)—R₇;
R₄ represents hydrogen or C₁₋₆alkyl;
R₅ represents hydrogen, C₁₋₆alkyl, hydroxyC₁₋₆alkyl, C₂₋₆alkenyl, C₂₋₆alynyl, polyhaloC₁₋₆alkyl, C₁₋₆alkyloxyC₁₋₆alkyl, aminoC₁₋₆alkyl, mono- or di(C₁₋₄alkyl)aminoC₁₋₆alkyl, aminocarbonylC₁₋₆alkyl mono- or di(C₁₋₄alkyl)aminocarbonylC₁₋₆alxyl or aryl;
R₆ₐ and R_{6b} each independently represent hydrogen, C₁₋₆alkyl, amino, mono- or di(C₁₋₄alkyl)amino, arylNH—, aminoC₁₋₆alkyl, mono- or di(C₁₋₄alkyl)amino-C₁₋₆alkyl, C₁₋₆alkylcarbonylamino, aminocarbonylamino, C₁₋₆alkyloxy, carbonylamino or hydroxyC₁₋₆alkyl; or
R₆ₐ and R_{6b} taken together with the nitrogen to which they are attached form pyrrolidinyl, imidazolidinyl, pyrazolidinyl, piperidinyl, piperazinyl, morpholinyl, thiomorpholinyl or piperazinyl substituted with C₁₋₆alkyl;
R₇ represents hydrogen, C₁₋₆alkyl, hydroxyC₁₋₆alkyl, C₂₋₆alkenyl, C₂₋₆alkynyl, polyhaloC₁₋₆alkyl C₁₋₆alkoxyC₁₋₆alkyl, aminoC₁₋₆alkyl, mono- or di(C₁₋₄alkyl)aminoC₁₋₆alkyl, aminocarbonylC₁₋₆alkyl, mono- or di(C₁₋₄alkyl)aminocarbonylC₁₋₆alkyl, aryl or heteroaryl;
Z represents a cyclic ring system selected from 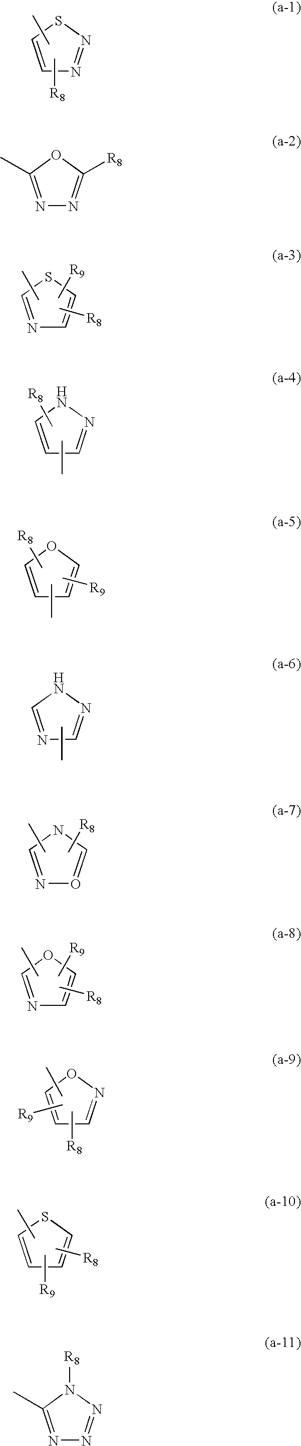 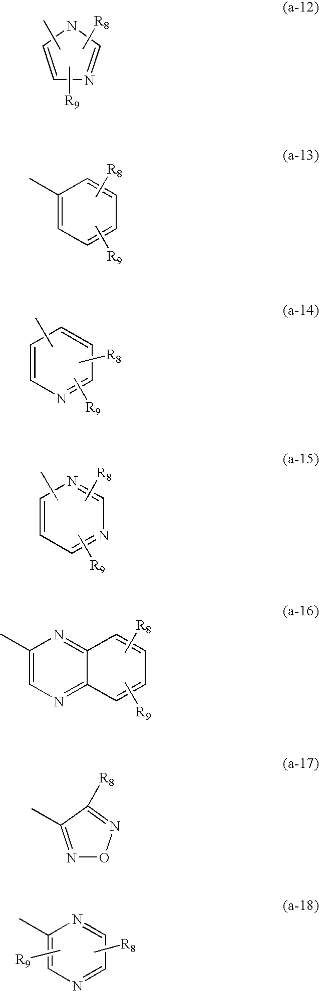
each R₈ independently represents hydrogen, halo, C₁₋₆alkyl, C₁₋₆alkyloxy, polyhaloC₁₋₆alkyl, polyhaloC₁₋₆alkyloxy; cyano, aminocarbonyl, mono- or di(C₁₋₄alkyl)aminocarbonyl, amino, mono- or di(C₁₋₄alkyl)amino, hydroxyC₁₋₆alkylamino, aryl, aryloxy, piperidinyl, piperidinylamino, morpholinyl, piperazinyl or nitro;
each R₉ independently represents hydrogen, halo or C₁₋₆alkyl;
n is 1, 2, 3, 4 or 5;
aryl represents phenyl or phenyl substituted with one, two, three, four or five substituents each independently selected from halo, C₁₋₆alkyl, C₁₋₆alkyloxy, polyhaloC₁₋₆alkyl, polyhaloC₁₋₆alkyloxy, cyano, aminocarbonyl, mono- or di(C₁₋₄alklyl)aminocarbonyl, amino, mono- or di(C₁₋₄alkyl)amino, phenyloxy or nitro;
heteroaryl represents furanyl, thienyl, pyrrolyl, oxazolyl, thiazolyl, imidazolyl, pyrazolyl, isoxazolyl, isothiazolyl, oxadiazolyl, triazolyl, thiadiazolyl, pyridyl, pyridazinyl, pyrimidinyl, pyrazinyl, each of said heterocycles optionally being substituted with one or two substituents each independently selected from halo, C₁₋₆alkyl, C₁₋₆alkyloxy, polyhaloC₁₋₆alkyl, polyhaloC₁₋₆alkyloxy, cyano, aminocarbonyl, mono- or di(C₁₋₄alkyl)aminocarbonyl, amino, mono- or di(C₁₋₄alkyl)amino or nitro.

A second interesting embodiment are those compounds of formula (I) or any subgroup thereof as mentioned hereinbefore as interesting embodiment wherein R₃ represents hydrogen, cyano, C(═O)—O—R₅, C(═O)—NR₆ₐR_{6b}, C(═S)—NR₆ₐR_{6b}, S(═O)₂—NR₆ₐR_{6b} or C(═O)—R₇.

A third interesting embodiment are the compounds of formula (I) or any subgroup thereof as mentioned hereinbefore as interesting embodiment wherein R₃ represents cyano, C₁₋₆alkyl optionally substituted with hydroxy or C₁₋₆alkyloxy, C(═O)—O—R₅, C(═O)—NR₆ₐR_{6b}, C(═S)—NR₆ₐR_{6b}, S(═O)₂—NR₆ₐR_{6b} or C(═O)—R₇.

A fourth interesting embodiment are the compounds of formula (I) or any subgroup thereof as mentioned hereinbefore as interesting embodiment wherein R₃ represents cyano, C(═O)—O—R₅, C(═O)—NR₆ₐR_{6b}, C(═S)—NR₆ₐR_{6b}, S(═O)₂—NR₆ₐR_{6b} or C(═O)—R₇.

A fifth interesting embodiment are the compounds of formula (I) or any subgroup thereof as mentioned hereinbefore as interesting embodiment wherein R₃ represents hydrogen, cyano, C(═O)—O—R₅, C(═O)—NR₆ₐR_{6b} or C(═O)—R₇; preferably hydrogen, C₁₋₆alkyl substituted with C₁₋₆alkyloxy, or C(═O)—O—R₅; more preferably hydrogen or C(═O)—O—R₅; even more preferably C(═O)—O—R₅, in particular C(═O)—O—C₁₋₆alkyl; most preferred C(═O)—O—CH₃.

A sixth interesting embodiment are those compounds of formula (I) or any subgroup thereof as mentioned hereinbefore as interesting embodiment wherein Z is other than 3-pyridyl.

A seventh interesting embodiment are those compounds of formula (I) or any subgroup thereof as mentioned hereinbefore as interesting embodiment wherein n is 2 or 3, in particular n is 2.

An eighth interesting embodiment are those compounds of formula (I) or any subgroup thereof as mentioned hereinbefore as interesting embodiment wherein n is 2 and said two substituents are placed in meta and para postion.

A ninth interesting embodiment are those compounds of formula (I) or any subgroup thereof as mentioned hereinbefore as interesting embodiment wherein Z is a cyclic ring system selected from (a-1), (a-2), (a-3), (a-4), (a-5), (a-6), (a-7), (a-9), (a-10), (a-11), (a-12), (a-13), (a-14), (a-15), (a-16) or (a-18); preferably a cyclic ring system selected from (a-2), (a-3), (a-4), (a-5), (a-6), (a-7), (a-11), (a-12), (a-13), (a-14) or (a-15); more preferably a cyclic ring system selected from (a-2), (a-3), (a-4), (a-5), (a-6), (a-7), (a-11), (a-13), (a-14) or (a-15); even more preferably a cyclic ring system selected from (a-2), (a-11) or (a-15); most preferred a cyclic ring system selected from (a-2) or (a-15). Also interesting are those compounds of formula (I) or any subgroup thereof as mentioned hereinbefore as interesting embodiment wherein Z is a cyclic ring system of (a-9).

A tenth interesting embodiment are those compounds of formula (I) or any subgroup thereof as mentioned hereinbefore as interesting embodiment wherein Z represents a cyclic ring system selected from (a-2) or (a-15) and R₃ represents hydrogen.

An eleventh interesting embodiment are those compounds of formula (I) or any subgroup thereof as mentioned hereinbefore as interesting embodiment wherein R₂ represents halo, C₁₋₆alkyl, C₁₋₆alkyloxy, polyhaloC₁₋₆alkyl or aryloxy, preferably halo, polyhaloC₁₋₆alkyl or aryloxy, more preferably halo or polyhaloC₁₋₆alkyl, in particular chloro, fluoro or trifluoromethyl, most preferred halo, in particular chloro or fluoro.

A twelfth interesting embodiment are the compounds of formula (I) or any subgroup thereof as mentioned hereinbefore as interesting embodiment wherein R₁ is hydrogen, methyl, ethyl, n-propyl, methoxymethyl, cyclohexyl, cyclopropyl, dimethylaminomethyl, 2-thienyl, 3,4-dichlorophenyl; preferably R₁ is C₁₋₆alkyl or C₁₋₆alkyloxyC₁₋₆alkyl, in particular methyl, ethyl, propyl, methoxymethyl, more in particular methyl, ethyl, n-propyl or methoxymethyl; more preferably R₁ is C₁₋₆alkyl, in particular methyl, ethyl and propyl, more in particular methyl, ethyl or n-propyl; most preferred R₁ is ethyl.

A thirteenth interesting embodiment are the compounds of formula (I) or any subgroup thereof as mentioned hereinbefore as interesting embodiment wherein R₄ is hydrogen.

A fourteenth interesting embodiment are those compounds of formula (I) or any subgroup thereof as mentioned hereinbefore as interesting embodiment which are stereochemically pure.

A fifteenth interesting embodiment are those compounds of formula (I) or any subgroup thereof as mentioned hereinbefore as interesting embodiment wherein the carbon atom carrying the R₁ and R₄ substituent has the (S) configuration, i.e. a compound of formula (I′) 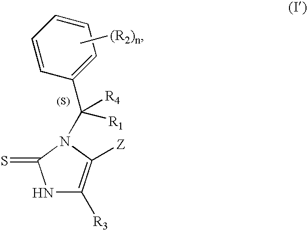
a N-oxide, a pharmaceutically acceptable addition salt, a quaternary amine or a stereochemically isomeric form thereof

Also interesting compounds are those compounds of formula (I) wherein one or more, preferably all of the following restrictions apply:
a) R₁ represents C₁₋₆alkyl or C₁₋₆alkyloxyC₁₋₆alkyl, especially methyl, ethyl, propyl or methoxymethyl;
b) R₂ represents halo, polyhaloC₁₋₆alkyl or aryloxy, especially halo, e.g. chloro or fluoro;
c) R₃ represents hydrogen, cyano, C(═O)—O—R₅, C(═O)—NR₆ₐR_{6b} or C(═O)—R₇;
d) Z represents a ring system selected from (a-1), (a-2), (a-3), (a-4), (a-5), (a-6), (a-7), (a-9), (a-10), (a-11), (a-12), (a-13), (a-14), (a-15), (a-16) or (a-18);
e) R₄ represents hydrogen;
f) n is 2 or 3.

Also interesting compounds consists of those compounds of formula (I) wherein one or more of the following restrictions apply
a) R₁ represents C₁₋₆alkyl, especially ethyl or n-propyl, more especially ethyl;
b) R₂ represents halo, polyhaloC₁₋₆alkyl or aryloxy, especially halo, e.g. chloro and fluoro;
c) R₃ represents hydrogen, C₁₋₆alkyl substituted with C₁₋₆alkyloxy, or C(═O)—O—R₅;
d) Z represents a ring system selected from (a-2), (a-3), (a-4), (a-5), (a-6), (a-7), (a-11), (a-13), (a-14), (a-15);
e) R₄ represents hydrogen;
f) n is 1, 2 or 3, in particular 2 or 3, more in particular 2.

Further interesting compounds consists of those compounds of formula (I) wherein one or more of the following restrictions apply:
a) R₁ represents C₁₋₆alkyl, especially ethyl;
b) R₂ represents halo, polyhaloC₁₋₆alkyl or aryloxy;
c) R₃ represents hydrogen;
d) Z represents a ring system selected from (a-2), (a-7) or (a-15) or (a-2), (a-11) or (a-15);
e) R₄ represents hydrogen;
f) n is 2 or 3, in particular 2.

Further interesting compounds consists of those compounds of formula (I) wherein one or more of the following restrictions apply:
a) R₁ represents C₁₋₆alkyl, especially ethyl;
b) R₂ represents halo, especially chloro or fluoro;
c) R₃ represents hydrogen;
d) Z represents a ring system selected from (a-2) or (a-15);
e) R₄ represents hydrogen;
f) n is 2 or 3, in particular 2.

Preferred compounds of formula (I) are compounds 5, 26, 1, 20, 21 and 9.

Most preferred compounds of formula (I) are compounds 1, 20, 21 and 9.

In general, compounds of formula (I) can be prepared by reacting an intermediate of formula (II) with an appropriate acid, such as hydrochloric acid or acetic acid, optionally in the presence of a suitable solvent, such as for example 1,4-dioxane.

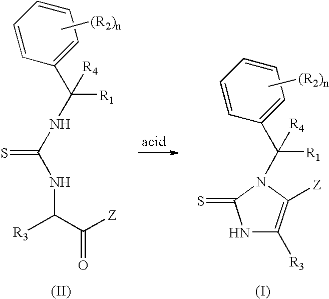

Intermediates of formula (I) wherein R₃ is hydrogen, said compounds being represented by formula (I-a), can be prepared by reacting an intermediate of formula (III) with an intermediate of formula (IV) in the presence of a suitable solvent, such as for example 1,4-dioxane and water.

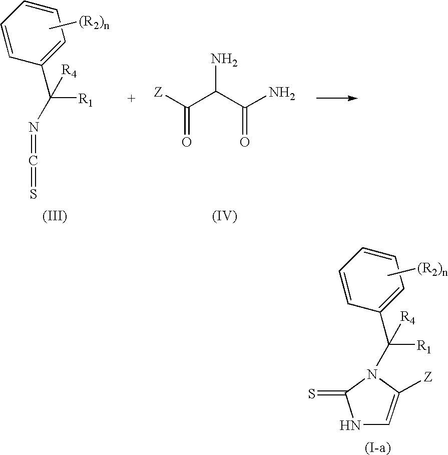

Compounds of formula (I) can also be prepared by reacting an intermediate of formula (V) with a suitable acid, such as for example trifluoroacetic acid.

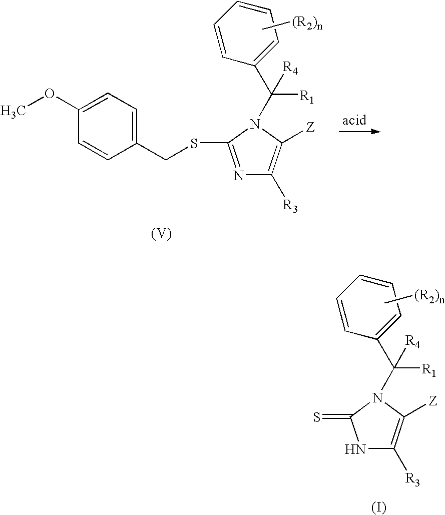

Compounds of formula (I) wherein Z represents optionally substituted 1,3,4-oxadiazole, said compounds being represented by formula (I-b), can be prepared by reacting an intermediate of formula (VI) with phosphoric trichloride (POCl₃) or Burgess'reagent in the presence of a suitable solvent, such as for example acetonitrile or tetrahydrofuran.

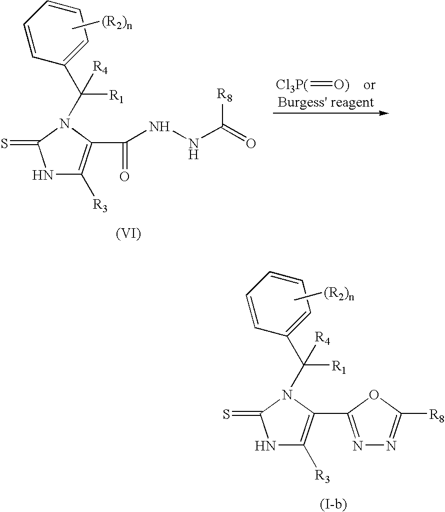

Compounds of formula (I) wherein Z represents 1,3,4-oxadiazole, said compounds being represented by formula (I-b1), can be prepared by reacting an intermediate of formula (VII) with SOCl₂ and HC(═O)NH—NH₂ in the presence of a suitable solvent, such as for example tetrahydrofuran.

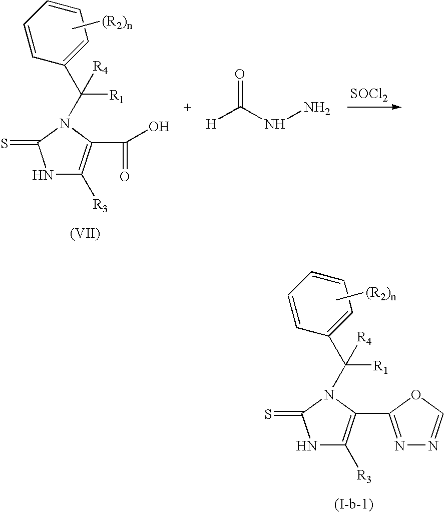

Compounds of formula (I) wherein Z represents optionally substituted 1,2,4-oxadiazole, said compounds being represented by formula (I-c), can be prepared by reacting an intermediate of formula (VII) with an intermediate of formula (IX) in the presence of a suitable base, such as for example NaOCH₃, and a suitable solvent, such as an alcohol, e.g. methanol.

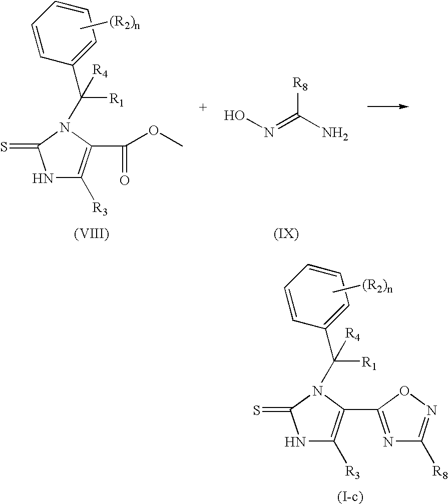

Compounds of formula (I) wherein Z represents tetrazolyl and wherein R₃ is hydrogen, said compounds being represented by formula (I-a-1), can be prepared by reacting an intermediate of formula X) with methyl formate in the presence of a suitable base, such as for example t-BuOK, NaOCH₃ or NaOC(CH₃)₃, KSCN in the presence of a suitable acid, such as for example hydrochloric acid and the like, and in the presence of a suitable solvent, such as for example tetrahydrofuran, an alcohol, such as for example methanol, and water, followed by reacting the thus obtained intermediate of formula (X-a) with Bu₃SnN₃ in the presence of a suitable solvent, such as for example toluene.

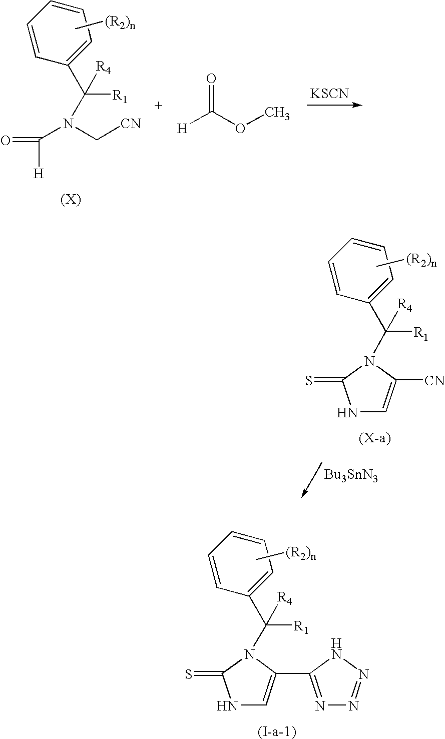

Compounds of formula (I′) can be prepared according to the above described reactions but starting from an intermediate wherein the carbon atom carrying the R₁ and R₄ substituen t has the (S) configuration.

Alternatively, compounds of formula (I) wherein the carbon atom carrying the R₁ and R₄ substituent has the (R) configuration can be prepared according to the above described reactions but starting from an intermediate wherein the carbon atom carrying the R₁ and R₄ substituent has the (R) configuration.

The compounds of formula (I) may further be prepared by converting compounds of formula (I) into each other according to art-known group transformation reactions.

The compounds of formula (I) may be converted to the corresponding N-oxide forms following art-known procedures for converting a trivalent nitrogen into its N-oxide form Said N-oxidation reaction may generally be carried out by reacting the starting material of formula (I) with an appropriate organic or inorganic peroxide. Appropriate inorganic peroxides comprise, for example, hydrogen peroxide, alkali metal or earth alkaline metal peroxides, e.g. sodium peroxide, potassium peroxide; appropriate organic peroxides may comprise peroxy acids such as, for example, benzenecarboperoxoic acid or halo substituted benzenecarboperoxoic acid, e.g. 3-chlorobenzenecarboperoxoic acid, peroxoalkanoic acids, e.g. peroxoacetic acid, alkylhydroperoxides, e.g. tert.butyl hydro-peroxide. Suitable solvents are, for example, water, lower alcohols, e.g. ethanol and the like, hydrocarbons, e.g. toluene, ketones, e.g. 2-butanone, halogenated hydrocarbons, e.g. dichloromethane, and mixtures of such solvents.

Compounds of formula (I) wherein R₃ represents C(═O)—O—C₁₋₆alkyl, may be converted into a compound of formula (I) wherein R₃ represents CH₂—OH by reaction with a suitable reducing agent, such as for example LiHBEt₃ in the presence of a suitable solvent, such as for example tetrahydrofuran.

Compounds of formula (I) wherein R₃ represents C(═O)—O—C₁₋₆alkyl, can also be converted into a compound of formula (I) wherein R₃ represents C(═O)—OH by reaction with a suitable base, such as NaOH, in the presence of a suitable solvent, such as for example H₂O, tetrahydrofuran or an appropriate alcohol, e.g. methanol and the like.

Compounds of formula (I) wherein R₃ represents C(═O)—O—C₁₋₆alkyl, can also be converted into a compound of formula (I) wherein R₃ represents C(═O)—NR₆ₐR_{6b}, by reaction with the appropriate base of formula NHR₆ₐR_{6b} in a suitable solvent, such as for example H₂O.

Compounds of formula (I) wherein R₃ represents C(═O)—O—H, can be converted into a compound of formula (I) wherein R₃ represents C(═O)—NR₆ₐR_{6b}, by reaction with the appropriate base of formula NHR₆ₐR_{6b} in the presence of N′-(ethylcarbonimidoyl)-NN-dimethyl-1,3-propanediamine, 1-hydroxy-1H-benzotriazole and a suitable solvent, such as for example N,N-dimethylformamide.

Compounds of formula (I) wherein R₃ represents C(═O)—O—H, can also be converted into a compound of formula (I) wherein R₃ represents C(═O)—NH₂ by reaction with NH₄OH in the presence of SOCl₂.

Compounds of formula (I) wherein R₃ represents C(═O)—O—C₁₋₆alkyl, can also be converted into a compound of formula (I) wherein R₃ represents C(═O)—O—C₁₋₆alkyl-O—C₁₋₆alkyl, by reaction with HO—C₁₋₆alkyl-O—C₁₋₆alkyl in the presence of NaBH₄.

Compounds of formula (I) wherein R₃ represents cyano or C(═O)—O—C₁₋₆alkyl, can be converted into a compound of formula (I) wherein R₃ represents aminocarbonyl by reaction with NH₄OH.

Compounds of formula (I) wherein R₃ represents cyano, can also be converted into a compound of formula (I) wherein R₃ represents C(═S)NR₆ₐR_{6b} by reaction with hydrogen sulfide in the presence of N-ethyl-N-(1-methylethyl)-2-propanamine in a suitable solvent such as pyridine.

Compounds of formula (I) wherein R₃ represents C(═O)—NR₆ₐR_{6b} can be converted into a compound of formula (I) wherein R₃ represents C(═O)—C₁₋₆alkyl by reaction with chloroC₁₋₆alkylMg in a suitable solvent such as tetrahydrofaran.

Compounds of formula (I) wherein R₃ represents C(═O)—C₁₋₆alkyl can be converted into compounds of formula (I) wherein R₃ represents hydroxyC₁₋₆alkyl by reaction with a suitable reducing agent such as NaBH₄, in the presence of a suitable solvent such as methanol.

Some of the compounds of formula (I) and some of the intermediates in the present invention may contain an asymmetric carbon atom. Pure stereochemically isomeric forms of said compounds and said intermediates can be obtained by the application of art-known procedures. For example, diastereoisomers can be separated by physical methods such as selective crystallization or chromatographic techniques, e.g. counter current distribution, liquid chromatography and the like methods. Enantiomers can be obtained from racemic mixtures by first converting said racemic mixtures with suitable resolving agents such as, for example, chiral acids, to mixtures of diastereomeric salts or compounds; then physically separating said mixtures of diastereomeric salts or compounds by, for example, selective crystallization or chromatographic techniques, e.g. liquid chromatography and the like methods; and finally converting said separated diastereomeric salts or compounds into the corresponding enantiomers. Pure stereochemically isomeric forms may also be obtained from the pure stereochemically isomeric forms of the appropriate intermediates and starting materials, provided that the intervening reactions occur stereospecifically.

An alternative manner of separating the enantiomeric forms of the compounds of formula (I) and intermediates involves liquid chromatography, in particular liquid chromatography using a chiral stationary phase.

Some of the intermediates and starting materials are known compounds and may be commercially available or may be prepared according to art-known procedures.

Intermediates of formula (II) can be prepared by reacting an intermediate of formula (III) with an intermediate of formula (XI) in the presence of a suitable base, such as for example dipotassium carbonate, and a suitable solvent, such as for example dioxane or tetrahydrofuiran and water.

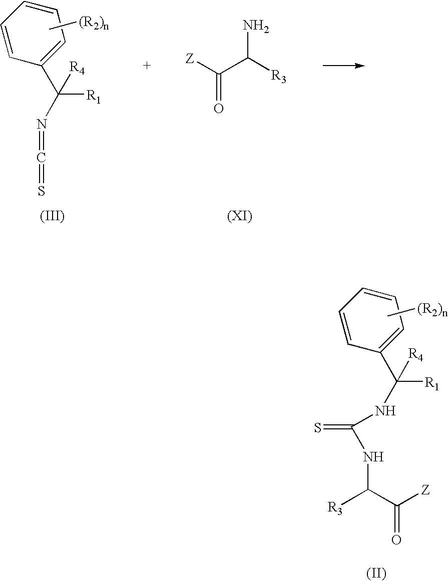

Intermediates of formula (III) can be prepared by reacting an intermediate of formula (XII) with C(═S)Cl₂ in the presence of a suitable base, such as for example N,N-diisopropylethanamine, and a suitable solvent, such as for example methylene chloride.

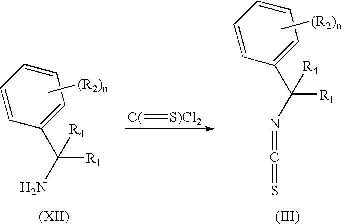

Intermediates of formula (XII) wherein R₄ represents hydrogen, said intermediates being represented by formula (XII-a), may be prepared by reacting an intermediate of formula (XIII) with a suitable reducing agent, such as for example H₂, in the presence of a suitable catalyst, such as for example Raney Nickel, optionally a suitable catalyst poison, such as for example a thiophene solution, a suitable base, such as for example NH_{3,} and a suitable solvent, such as for example an alcohol, e.g. methanol.

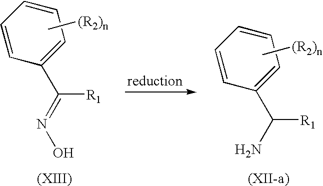

Intermediates of formula (XIII) may be prepared by reacting an intermediate of formula (XIV) with HO—NH₂ in the presence of a suitable base, such as for example Na₂CO₃ or sodium acetate and a suitable solvent, such as for example an alcohol, e.g. ethanol, and water.

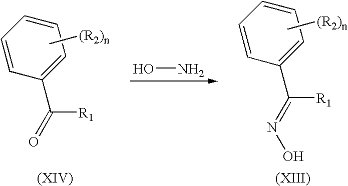

Intermediates of formula (XII) can be prepared as described hereinabove. The intermediates of formula (XII) may contain a chiral center at the carbon atom carrying the R₁ and R₄ substituent depending on the substituents representing R₁ and R₄. In case said carbon atom represents a chiral center, stereospecific intermediates of formula (XII) represented by formula (XII-b), can be prepared by reacting an intermediate of formula (XV) with triphenylphosphine, in the presence of a suitable solvent, such as for example tetrahydrofuran and water or by reacting an intermediate of formula (XV) with a suitable reducing agent, such as for example H₂, in the presence of a suitable catalyst, such as for example Pt on charcoal or Pd on charcoal, and a suitable solvent, such as for example an alcohol, e.g. methanol.

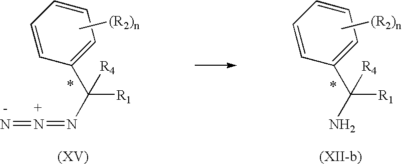

When a stereospecific intermediate of formula (XII-b) is reacted further according to the methods described hereinabove, the resulting intermediates are also stereospecific and finally the resulting final compounds are also stereospecific.

Intermediates of formula (XV) can be prepared by reacting an intermediate of formula (XVI) with diphenylphosphoryl azide in the presence of 2,3,4,6,7,8,9,10-octahydro-pyrimido[1,2-a]azepine and in the presence of a suitable solvent, such as for example toluene.

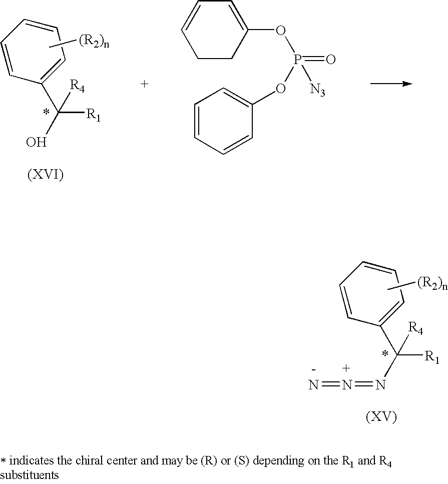

Stereospecific intermediates of formula (XVI) wherein R₄ is hydrogen and R₁ is methyl, ethyl, or n-propyl, said R₁ being represented by Alk and said intermediates being represented by formula (XVI-a) and (XVI-b), can be prepared by reacting an intermediate of formula (XVII) with ZnAlk₂ wherein Alk represents methyl, ethyl or n-propyl, in the presence of a stereospecific catalyst, such as for example N,N′-(1R,2R)-1,2-cyclohexanediylbis[1,1,1-trifluoro]-methanesulfonamide respectively N,N′-(1S,2S)-1,2-cyclohexanediylbis[1,1,1-trifluoro]-methanesulfonamide, Ti(iPrO)₄ and a suitable solvent, such as for example toluene.

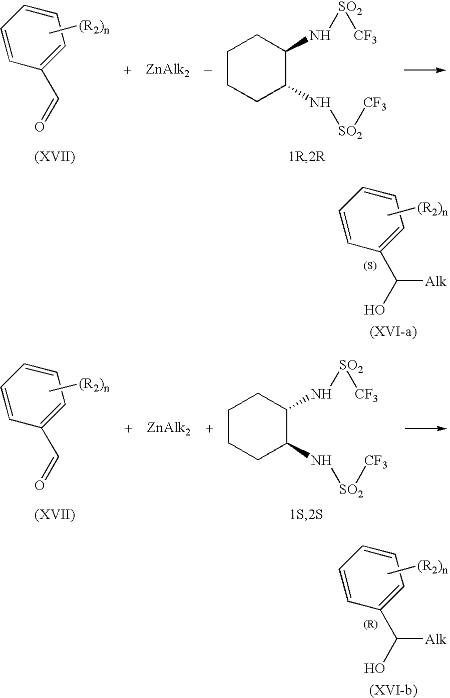

Intermediates of formula (V) wherein Z represents optionally substituted thiazolyl, said intermediates being represented by formula (V-a), can be prepared by reacting an intermediate of formula (XVIII) with an intermediate of formula (XIX) wherein W₁ represents a suitable leaving group, such as for example halo, e.g. chloro, bromo and the like, in the presence of a suitable solvent, such as for example an alcohol, e.g. ethanol.

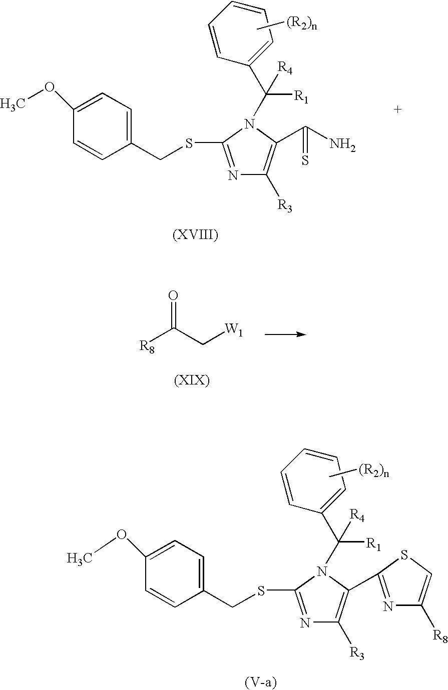

Intermediates of formula (XVIII) can be prepared by reacting an intermediate of formula (XX) with H₂S in the presence of a suitable base, such as for example N,N-diisopropylethanamine, and a suitable solvent, such as for example pyridine.

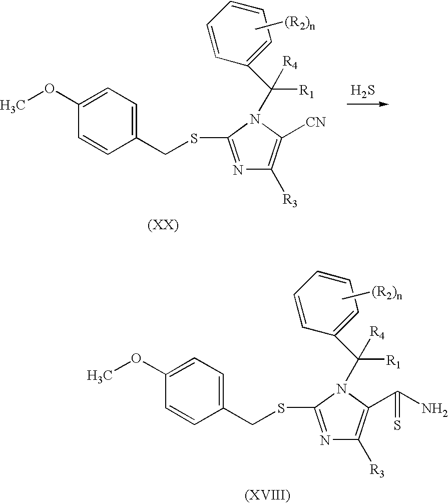

Intermediates of formula (XX) can be prepared by reacting an intermediate of formula (XXI) with 4-methoxy-benzenemethanol in the presence of a suitable acid, such as for example trifluoroacetic acid, and a suitable solvent, such as for example methylene chloride.

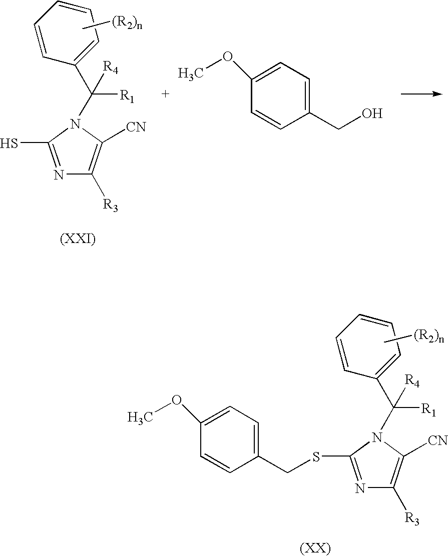

Intermediates of formula (XXI) can be prepared by reacting an intermediate of formula (X) with CH₃—CH(═O) in the presence of KSCN, in the presence of a suitable base, such as for example sodium methanolate, and a suitable solvent, such as for example tetrahydrofuran.

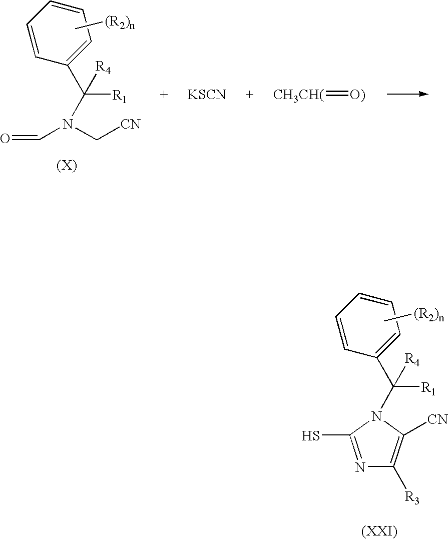

Intermediates of formula (X) can be prepared by reacting an intermediate of formula (XXII) with n-butyl formate.

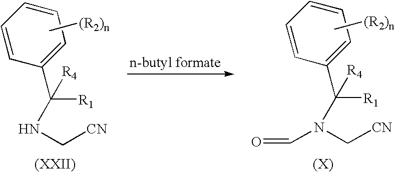

Intermediates of formula (XXII) can be prepared by reacting an intermediate of formula (XII) with an intermediate of formula (XXII) wherein W₂ represents a suitable leaving group, such as for example halo, e.g. chloro and the like, in the presence of a suitable base, such as for example N,N-diethylethanamine, and a suitable solvent, such as for example N,N-dimethylformamide or tetrahydrofuran.

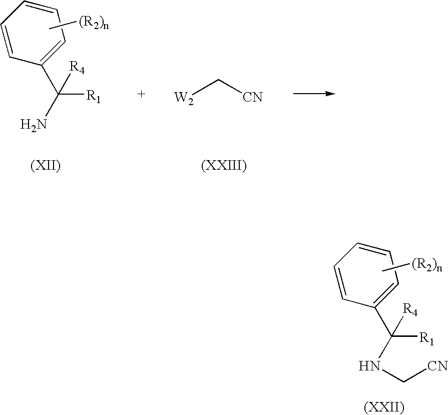

Intermediates of formula (V) wherein Z represents pyrazolyl or optionally substituted pyrimidine, said intermediates being represented by formula (V-b) or (V-c), can be prepared by reacting an intermediate of formula (XXIV) with NH₂—NH₂ or R₈—C(═NH)NH₂ in the presence of Na and a suitable solvent, such as for example an alcohol e.g. ethanol.

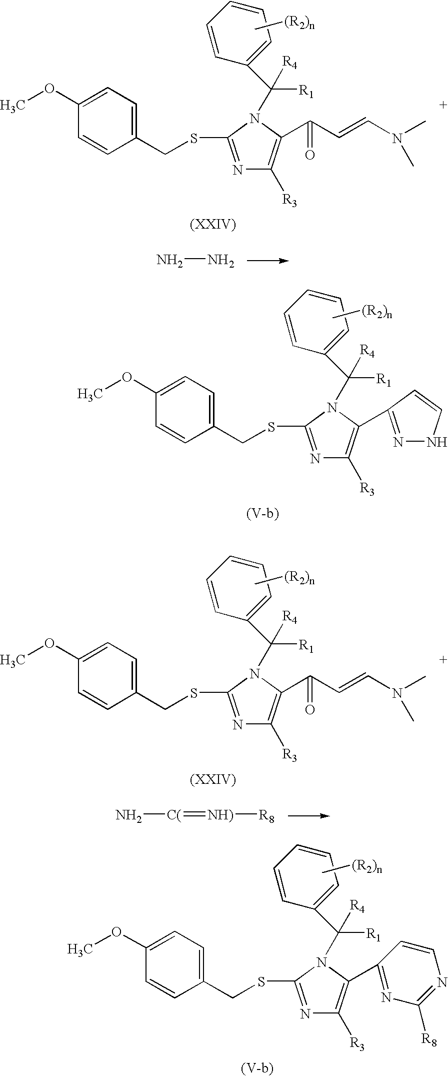

Intermediates of formula (XXV) can be prepared by reacting an intermediate of formula (XXV) with N(CH₃)₂—CH(OCH₃)₂.

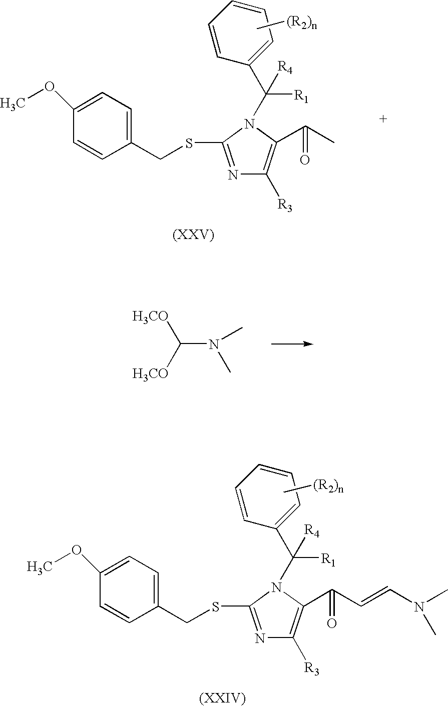

Intermediates of formula (XXV) can be prepared by reacting an intermediate of formula (XXVI) with an intermediate of formula (XXVII) wherein W₃ represents a suitable leaving group, such as for example halo, e.g. chloro, in the presence of NaH and a suitable solvent, such as for example tetrahydrofuran.

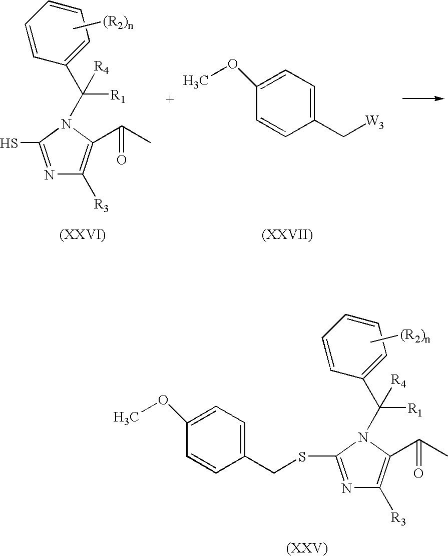

Intermediates of formula (XXVI) can be prepared by reacting an intermediate of formula (XXVIII) with CH₃MgCl in the presence of a suitable solvent, such as for example tetrahydrofuran.

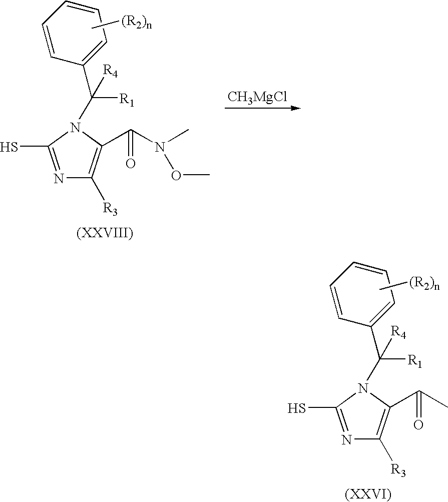

Intermediates of formula (XXVIII) can be prepared by reacting an intermediate of formula (XXIX) wherein W₄ represents a suitable leaving group, such as for example halo, e.g. chloro and the like, with NH(CH₃)(OCH₃) in the presence of a suitable base, such as for example N,N-diethylethanamine, and a suitable solvent, such as for example tetrahydrofuran.

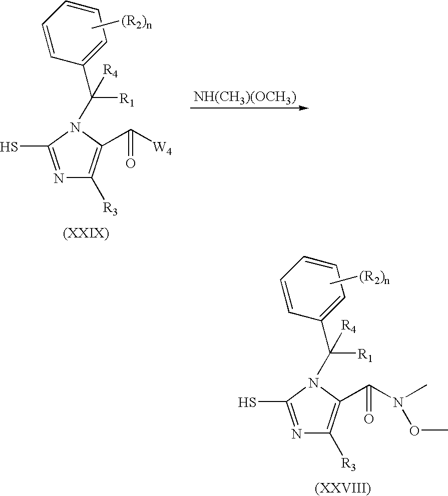

Intermediates of formula (XXIX) wherein W₄ represents chloro, said intermediates being represented by formula (XXIX-a), can be prepared by reacting an intermediate of formula (VII) with SOCl₂.

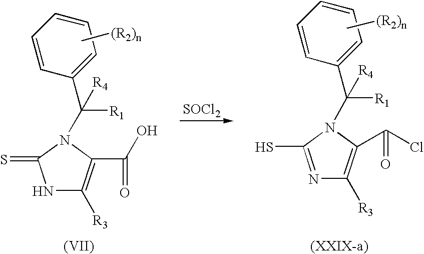

Intermediates of formula (VII) can be prepared by reacting an intermediate of formula (VIII) with a suitable base, such as for example sodium hydroxide, in the presence of a suitable solvent, such as for example an alcohol, e.g. methanol, or tetrahydrofuran.

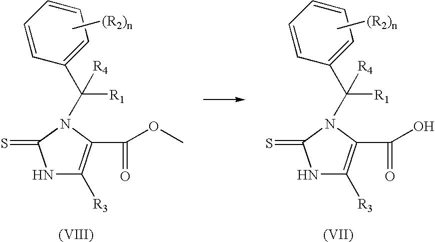

Intermediates of formula (VIII) wherein R₃ is hydrogen, said intermediates being represented by formula (VIII-a), can be prepared by reacting an intermediate of formula (XXX) with formic acid methyl ester or acetic acid methyl ester in the presence of KSCN, a suitable base, such as for example NaOCH₃ or t-BuONa, and a suitable solvent, such as for example tetrahydrofuran or an alcohol, e.g. methanol.

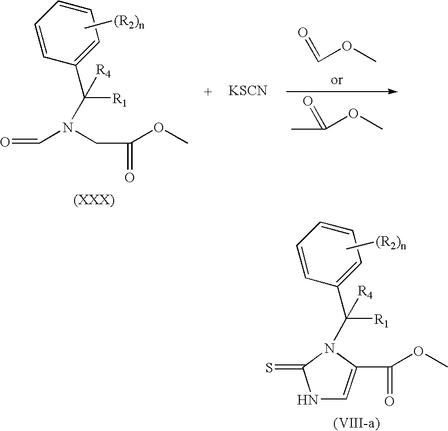

Intermediates of formula (XXX) can be prepared from an intermediate of formula (XXXI) in the presence of formic acid or formate, such as for example n-butylformate, and in the presence of a suitable solvent, such as for example xylene or diethyl ether.

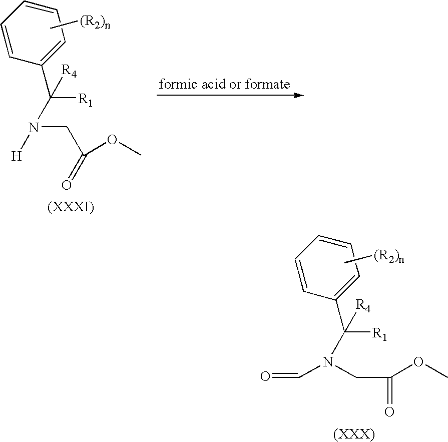

Intermediates of formula (XXXI) can be prepared by reacting an intermediate of formula (XII) with an intermediate of formula (XXXII) wherein W₅ represents a suitable leaving group, such as for example halo, e.g. chloro or bromo, in the presence of a suitable solvent, such as for example tetrahydrofuran or N,N-dimethylformamide, and optionally in the presence of a suitable base, such as for example N,N-diethylethanamine and NAN-dimethyl-4-pyridinamine.

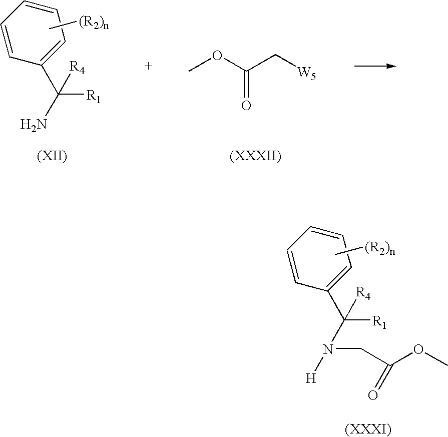

Intermediates of formula (V) wherein Z represents triazolyl, said intermediates being represented by formula (V-d), can be prepared by reacting an intermediate of formula (XXXIII) with NH₂—NH₂ in the presence of a suitable acid, such as for example acetic acid.

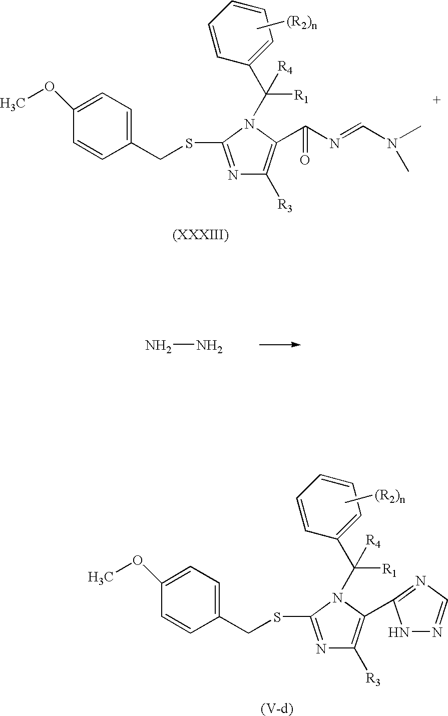

Intermediates of formula (XXXIII) can be prepared by reacting an intermediate of formula (XXXIV) with N,N-dimethylformamide/N,N-diinethylacetamide.

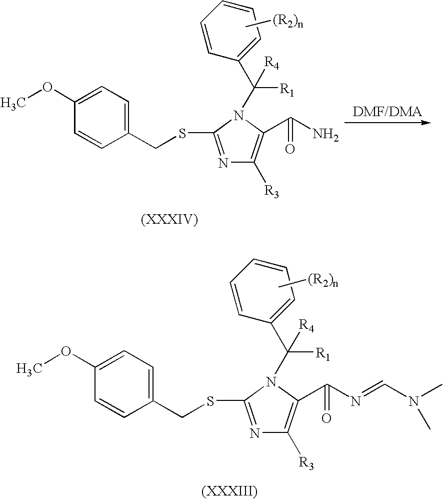

Intermediates of formula (XXXIV) can be prepared by reacting an intermediate of formula (XXXV) wherein W₆ represents a suitable leaving group, such as for example halo, e.g. chloro, with NH₃ in the presence of a suitable solvent, such as for example tetrahydrofuran.

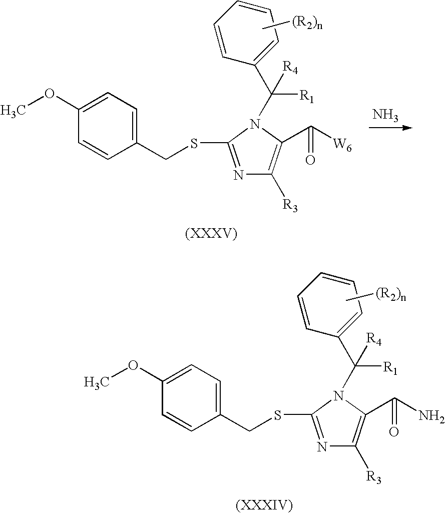

Intermediates of formula (XXXV) wherein W₆ represents chloro, said intermediates being represented by formula (XXXV-a), can be prepared by reacting an intermediate of formula (XXXVI) with SOCl₂.

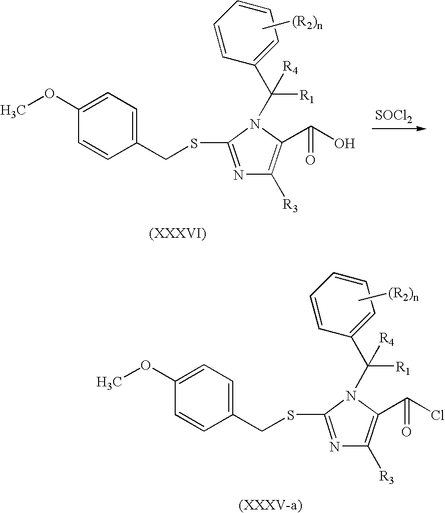

Intermediates of formula (XXXVI) can be prepared by hydrolysis of the corresponding ester (XXXVII) with a suitable base, such as for example sodium hydroxide, in the presence of a suitable solvent, such as for example an alcohol, e,g. methanol.

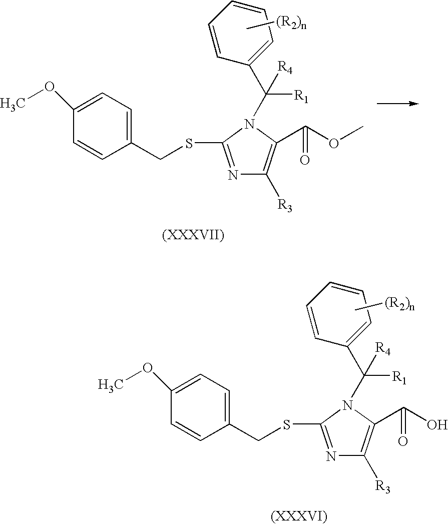

Intermediates of formula (XXXVII) can be prepared by reacting an intermediate of formula (XXIII) with 4-methoxy-benzenemethanol in the presence of a suitable acid, such as for example trifluoroacetic acid, and a suitable solvent, such as for example methylene chloride.

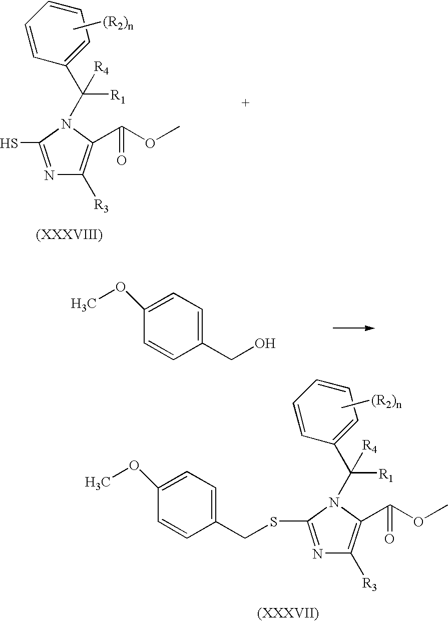

Intermediates of formula (VI) can be prepared by reacting an intermediate of formula (XXIX) or (VII) with an intermediate of formula (XXXIX) in the presence of a suitable base, such as for example N,N-diisopropylethanamine, a suitable diimide, such as for example N′-(ethylcarbonimidoyl)-N,N-dimethyl-1,3-propanediamine or dicyclohexylcarbondiimide, 1-hydroxy-1H-benzotriazole and a suitable solvent, such as for example tetrahydrofuran or N,N-dimethylformamide.

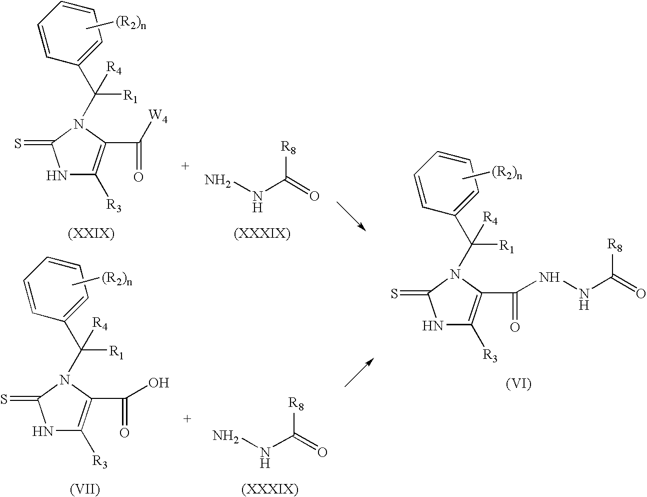

In the preparation of the compounds of the present invention, interesting intermediates are intermediates of formula (XII) 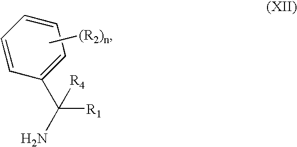
a N-oxide, a pharmaceutically acceptable addition salt, a quaternary amine or stereochemically isomeric form thereof.

As already indicated hereinabove, the intermediates of formula (XII) may contain a chiral center at the carbon atom carrying the R₁ and R₄ substituent depending on the substituents representing R₁ and R₄. In case said carbon atom represents a chiral center, a preferred embodiment of the intermediates of formula (XII) are those intermediates wherein the intermediate is stereospecific, i.e. wherein the intermediate has the (R) or (S) configuration at the carbon atom carrying the R₁ and R₄ substituent (intermediates of formula (XII-b). Particularly preferred are those intermediates of formula (XII-b) which have the (S) configuration (intermediates of formula (XII-b-1).

Thus the present invention also relates to intermediates of formula (XII-b-1) 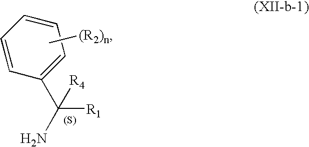
a N-oxide, a pharmaceutically acceptable addition salt or a quaternary amine thereof.

The present invention also relates to intermediates of formula (XII-b-1) provided that when n=2 and each R₂ is chloro and said two chloro substituents are placed in meta and para position, then R₁ is other than ethyl.

Another preferred embodiment are those intermediates of formula (XII-b-1) wherein each R₂ is independently selected from halo, C₁₋₆alkyl, C₁₋₆alkyloxy or polyhaloC₁₋₆alkyl provided that when n=2 and each R₂ is chloro and said two chloro substituents are placed in meta and para position, then R₁ is other than ethyl.

A further embodiment are those intermediates of formula (XII-b-1) wherein n is 1, 2, or 3, in particular 2 and provided that when n is 2 and each R₂ is chloro and said two chloro substituents are placed in meta and para position, then R₁ is other than ethyl.

Again another embodiment are those intermediates of formula (XII-b-1) wherein n is 2 and the two R₂ substituents are placed in meta and para position provided that when each R₂ is chloro, then R₁ is other than ethyl.

Another embodiment are those intermediates of formula (XII-b-1) as described hereinabove wherein R₄ is hydrogen.

Also interesting are those intermediates of formula (XII) and (XII-b-1) wherein R₁ is hydrogen, methyl, ethyl, n-propyl, methoxymethyl, cyclohexyl, cyclopropyl, dimethylaminomethyl, 2-thienyl, 3,4-dichlorophenyl, in particular methyl, ethyl, n-propyl, methoxymethyl, more in particular methyl, ethyl and n-propyl provided that when n is 2 and R₂ represents chloro, and said two chloro substituents are placed in meta and para position and R₄ is hydrogen, then R₁ is other than ethyl, cyclopropyl phenyl, and provided that when n is 2 and R₂ represents chloro, and said two chloro substituents are placed in meta and para position and R₄ is methyl, then R₁ is other than methyl and provided that when n is 2 and said two R₂ substituents are placed in meta and para position and R₂ in meta position is trifluoromethyl and R₂ in para position is fluoro and R₄ is hydrogen, then R₁ is other than ethyl.

Also interesting are those intermediates of formula (XII) or (XII-b-1) as described hereinabove wherein R₂ is chloro, fluoro, or trifluoromethyl, in particular chloro.

Particularly interesting intermediates are those intermediates of formula (XII-a) or (XII-b-1) having the following formula 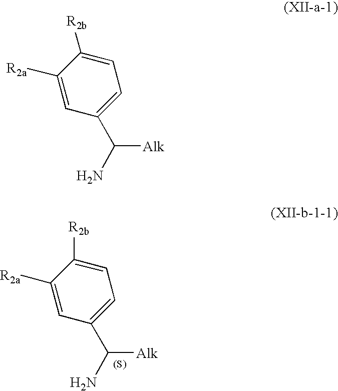
a N-oxide, a pharmaceutically acceptable addition salt or a quaternary amine thereof, wherein Alk is defined as hereinabove, i.e. Alk represents methyl, ethyl and n-propyl, and each R₂ₐ and R_{2b} independently represents chloro, fluoro, trifluoromethyl.

An interesting embodiment are those intermediates of formula (XII-a-1) provided that when R₂ₐ and R_{2b} are both chloro or when R₂ₐ is trifluoromethyl and R_{2b} is fluoro, then Alk is other than ethyl.

Further interesting intermediates of formula (XII-a-1) are those intermediates of formula (XII-a-1) provided that when R₂ₐ and R_{2b} are both chloro, then Alk is other than methyl, ethyl, n-propyl and provided that when R₂ₐ and R_{2b} are both fluoro or R₂ₐ is trifluoromethyl and R_{2b} is fluoro or R₂ₐ is fluoro and R_{2b} is trifluoromethyl then Alk is other than ethyl.

Also interesting are those intermediates of formula (XII-b-1-1) provided that when R₂ₐ and R_{2b} are both chloro, then Alk is other than ethyl.

The compounds of formula (I) and (I′) show CCR2 receptor antagonistic properties.

The C—C chemokine receptor 2 (CCR₂) and its ligand monocyte chemoattractant (chemotactic) protein (MCP-1; in new chemokine nomenclature also called CCL2) are recognized to be implicated in both acute and chronic inflammatory processes.

Chemokines (contraction of “chemotactic cytokines”) are most important regulators of leukocyte trafficking. This biological role is exerted by interacting—on target cells—with seven-transmembrane-domain receptors that are coupled to heterodimeric G proteins. Chemokines are mainly grouped into 2 major families (C—C or C—X—C family) dependent on the presence of an amino acid (represented by X) between the two conserved cysteine residues (represented by C) near the amino terminus. In general, chemokines from the C—C family attract monocytes, macrophages, T cells and NK cells.

A chemokine, which acts through the CCR2 receptor, is MCP-1 as indicated above. Therefore, the CCR2 receptor is also known as the MCP-1 receptor. MCP-2, MCP-3 and MCP-4 may also act, at least in part, through this receptor.

It is recognized that the CCR2 receptor and MCP-1 play a role in the pathophysiology of various inflammatory diseases. Therefore, CCR2 receptor antagonists, which block the CCR2 receptor, have potential as pharmaceutical agents to combat inflammatory conditions such as arthritis, osteoarthritis, rheumatoid arthritis, glomerulonephritis, diabetic nephropathy, lung fibrosis, idiopathic pulmonary fibrosis, sarcoidosis, vasculitis, hepatitis, nonalcoholic steatohepatitis, inflammatory conditions of the brain such as Alzheimer's disease, restenosis, alveolitis, asthma, allergic rhinitis, allergic conjunctivitis, atherosclerosis, psoriasis, delayed-type hypersensitivity reactions of the skin, inflammatory bowel disease, acute or chronic brain inflammation, e.g. multiple sclerosis, autoimmune encephalomyelitis, chronic obstructive pulmonary disease (COPD), uveitis, dermatitis, atopic dermatitis. CCR2 receptor antagonists may also be useful to treat autoimmune diseases such as diabetes or transplant rejection, stroke, reperfusion injury, ischemia, cancer, myocardial infraction, pain, in particular neuropathic pain.

The compounds of the present invention may also be used to inhibit the entry of Human Immunodeficiency Virus (HIV) into monocytes and lymphocytes, thereby having a therapeutic role in the treatment of AIDS (Acquired Immunodeficiency Syndrome).

The CCR2 receptor exists in two isoforms, namely the CCR2A and the CCR2B receptor.

Due to their CCR2 receptor antagonistic activity, in particular their CCR2B receptor antagonistic activity, the compounds of formula (I), their N-oxides, pharmaceutically acceptable addition salts, quaternary amines or stereochemically isomeric forms are useful in the treatment or prevention, in particular for the treatment, of diseases or conditions mediated through the activation of the CCR2 receptor, in particular the CCR2B receptor. Diseases or conditions related to an activation of the CCR2 receptor comprise inflammatory conditions such as arthritis, osteoarthritis, rheumatoid arthritis, glomerulonephritis, diabetic nephropathy, lung fibrosis, idiopathic pulmonary fibrosis, sarcoidosis, vasculitis, hepatitis, nonalcoholic steatohepatitis, inflammatory conditions of the brain such as Alzheimer's disease, restenosis, alveolitis, asthma, allergic rhinitis, allergic conjunctivitis, atherosclerosis, psoriasis, delayed-type hypersensitivity reactions of the skin, inflammatory bowel disease, acute or chronic brain inflammation, e.g. multiple sclerosis, autoimmune encephalomyelitis, chronic obstructive pulmonary disease (COPD), uveitis, dermatitis, atopic dermatitis, autoimmune diseases such as diabetes or transplant rejection, stroke, reperfusion injury, ischemia, cancer, myocardial infraction, pain (neuropathic pain). In particular, the compounds of formula (I) are useful in the treatment or prevention of inflammatory diseases and autoimmune diseases, especially rheumatoid arthritis, atherosclerosis, multiple sclerosis, inflammatory bowel disease and chronic obstructive pulmonary disease (COPD). The compounds of formula (I) are also of particular interest in the treatment or prevention of psoriasis, asthma, rheumatoid arthritis or pain (neuropathic pain), more in particular psoriasis, asthma or rheumatoid arthritis.

In view of the above-described pharmacological properties, the compounds of formula (I), their N-oxides, pharmaceutically acceptable addition salts, quaternary amines and stereochemically isomeric forms, may be used as a medicine. In particular, the present compounds can be used for the manufacture of a medicament for treating or preventing diseases mediated through activation of the CCR2 receptor, in particular the CCR2B receptor. More in particular, the compounds of the invention can be used for the manufacture of a medicament for treating or preventing inflammatory diseases, especially rheumatoid arthritis, atherosclerosis, multiple sclerosis, inflammatory bowel disease and chronic obstructive pulmonary disease (COPD). The compounds of the invention can also in particular be used for the manufacture of a medicament for treating or preventing psoriasis, asthma, rheumatoid arthritis or pain (neuropathic pain), more in particular psoriasis, asthma or rheumatoid arthritis.

In view of the utility of the compounds of formula (I), there is provided a method of treating warm-blooded animals, including humans, suffering from or a method of preventing warm-blooded animals, including humans, to suffer from diseases mediated through activation of the CCR2 receptor, in particular mediated through the CCR2B receptor. Said methods comprise the administration of an effective amount of a compound of formula (I), a N-oxide form, a pharmaceutically acceptable addition salt, a quaternary amine, or a possible stereoisomeric form thereof, to warm-blooded animals, including humans.

The blockade of the CCR2 receptor by the present compounds of formula (I) inhibits the normal function of MCP-1. Therefore, the present compounds can also be described as MCP-1 inhibitors and hence can be used to prevent or treat diseases mediated through MCP-1.

The present invention also provides compositions for preventing or treating diseases mediated through activation of the CCR2 receptor, in particular the CCR2B receptor. Said compositions comprise a therapeutically effective amount of a compound of formula (I) and a pharmaceutically acceptable carrier or diluent.

The compounds of the present invention may be formulated into various pharmaceutical forms for administration purposes. As appropriate compositions there may be cited all compositions usually employed for systemically administering drugs. To prepare the pharmaceutical compositions of this invention, an effective amount of the particular compound, optionally in addition salt form, as the active ingredient is combined in intimate admixture with a pharmaceutically acceptable carrier, which carrier may take a wide variety of forms depending on the form of preparation desired for administration. These pharmaceutical compositions are desirable in unitary dosage form suitable, particularly, for administration orally, rectally, percutaneously, or by parenteral injection. For example, in preparing the compositions in oral dosage form, any of the usual pharmaceutical media may be employed such as, for example, water, glycols, oils, alcohols and the like in the case of oral liquid preparations such as suspensions, syrups, elixirs, emulsions and solutions; or solid carriers such as starches, sugars, kaolin, diluents, lubricants, binders, disintegrating agents and the like in the case of powders, pills, capsules, and tablets. Because of their ease in administration, tablets and capsules represent the most advantageous oral dosage unit forms, in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. Also included are solid form preparations, which are intended to be converted, shortly before use, to liquid form preparations. In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot-on, as an ointment.

The compounds of the present invention may also be administered via inhalation or insufflation by means of methods and formulations employed in the art for administration via this way. Thus, in general the compounds of the present invention may be administered to the lungs in the form of a solution, a suspension or a dry powder. Any system developed for the delivery of solutions, suspensions or dry powders via oral or nasal inhalation or insufflation are suitable for the administration of the present compounds.

The compounds of the present invention may also be topically administered in the form of drops, in particular eye drops. Said eye drops may be in the form of a solution or a suspension. Any system developed for the delivery of solutions or suspensions as eye drops are suitable for the administration of the present compounds.

It is especially advantageous to formulate the aforementioned pharmaceutical compositions in unit dosage form for ease of administration and uniformity of dosage. Unit dosage form as used herein refers to physically discrete units suitable as unitary dosages, each unit containing a predetermined quantity of active ingredient calculated to produce the desired therapeutic effect in association with the required pharmaceutical carrier. Examples of such unit dosage forms are tablets (including scored or coated tablets), capsules, pills, powder packets, wafers, suppositories, injectable solutions or suspensions and the like, and segregated multiples thereof.

The exact dosage and frequency of administration depends on the particular compound of formula (I) used, the particular condition being treated, the severity of the condition being treated, the age, weight, sex, extent of disorder and general physical condition of the particular patient as well as other medication the individual may be taking, as is well known to those skilled in the art. Furthermore, it is evident that said effective daily amount may be lowered or increased depending on the response of the treated subject and/or depending on the evaluation of the physician prescribing the compounds of the instant invention.

The compounds of formula (I) may also be used in combination with other conventional anti-inflammatory or immunosuppressive agents, such as steroids, cyclooxygenase-2 inhibitors, non-steroidal-anti-inflammatory drugs, TNF-α antibodies, such as for example acetyl salicylic acid, bufexamac, diclofenac potassium, sulindac, diclofenac sodium, ketorolac trometamol, tolmetine, ibuprofen, naproxen, naproxen sodium, tiaprofen acid, flurbiprofen, mefenamic acid, nifluminic acid, meclofenamate, indomethacin, proglumetacine, ketoprofen, nabumetone, paracetamol, piroxicam, tenoxicam, nhnesulide, fenylbutazon, tramadol, beclomethasone dipropionate, betamethasone, beclamethasone, budesonide, fluticasone, mometasone, dexamethasone, hydrocortisone, methylprednisolone, prednisolone, prednisone, triamcinolone, celecoxib, rofecoxib, valdecoxib, infliximab, leflunomide, etanercept, CPH 82, methotrexate, sulfasalazine, antilymphocytory inmunoglobulines, antithymocytory immunoglobulines, azathioprine, cyclosporine, tacrolimnus substances, ascomycin, rapamycin, muromonab-CD3.

Thus, the present invention also relates to the combination of a compound of formula (I) and another anti-inflammatory or immunosuppressive agent. Said combination may be used as a medicine. The present invention also relates to a product containing (a) a compound of formula (I), and (b) another anti-inflammatory or immunosuppressive compound, as a combined preparation for simultaneous, separate or sequential use in the treatment of diseases mediated through activation of the CCR2 receptor, in particular mediated through the CCR2B receptor. The different drugs in such products may be combined in a single preparation together with pharmaceutically acceptable carriers. Alternatively, such products may comprise, for example, a kit comprising a container with a suitable composition containing a compound of formula (I) and another container with a composition containing another anti-inflammatory or immunosuppressive compound. Such a product may have the advantage that a physician can select on the basis of the diagnosis of the patient to be treated the appropriate amounts of each component and the sequence and timing of the administration thereof.

The following examples are intended to illustrate the present invention.

Exiperimental Part

Hereinafter, “THF” means tetrahydrofuran, “DIPE” means diisopropylether, “DMF” means N;N-dimethylformamide and “DMA” means N,N-dirnethylacetamide.

A. Preparation of the Intermediate Compounds

### EXAMPLE A1

a. Preparation of Intermediate 1

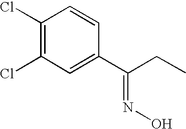

A solution of Na₂CO₃ (part of 0.52 mol) in H₂O (150 ml) was added to a stirring mixture of 1-(3,4-dichlorophenyl)-1-propanone (0.345 mol) in ethanol, p.a. (150 ml), then the remainder of Na₂CO₃ was added and hydroxylamine monohydrochloride (0.345 mol) was added portionwise while stirring vigorously. The reaction mixture was heated to reflux temperature and extra H₂O (75 ml) was added, then the resulting mixture was stirred and refluxed for 6 hours. Extra hydroxylamine monohydrochloride (2.4 g) was added and the mixture was refluxed further for 18 hours. Again extra hydroxylamine monohydrochloride (3 g) was added; the reaction mixture was refluxed for 24 hours and stirred for 2 days at room temperature. The solids were filtered off, washed with EtOH/H₂O (1/1) and dried (vacuum, stream of air) at 56° C. Yield: 71.8 g of intermediate 1 (95.4%).
b. Preparation of Intermediate 2 and 3

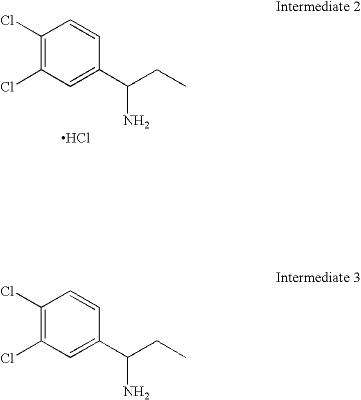

A mixture of intermediate 1 (0.3 mol) in CH₃OH/NH₃ (7 N) (500 ml) was hydrogenated at 14° C. with Raney Nickel (catalytic quantity) as a catalyst in the presence of thiophene (6 ml). After uptake of H₂ (2 equiv.), the catalyst was filtered off and the filtrate was evaporated, then co-evaporated 2 times with toluene. The residue was stirred in boiling 2-propanol (250 ml) and the mixture was filtered off hot. The filtrate was allowed to reach room temperature and HCl/2-propanol (6N, 150 ml) was added slowly while stirring vigorously. The solvent was evaporated and the residue was stirred in DIPE, then filtered off, washed and dried (vacuum) at 60° C. Yield: 53 g intermediate 2 (73.4%). A part of this fraction was converted into its free base: Intermediate 2 (18.0 g) was stirred in CH₂Cl₂ (200 ml) and a 15% aqueous K₂CO₃ solution was added, then the resulting mixture was stirred for 1 hour and a 50% NaOH solution was added to increase the pH. The organic layer was separated, washed with H₂O, dried (MgSO₄), filtered off and the solvent was evaporated. Yield: 12.4 g of intermediate 3.
c. Preparation of Intermediate 4

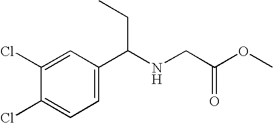

A solution of intermediate 2 (prepared according to A1.b) (0.0748 mol) and chloro acetic acid methyl ester (0.08 mol) in DMF, p.a., dried on molecular sieves, (150 ml) was stirred at room temperature under N₂ and Et₃N (0.224 mol) was slowly added, then the reaction mixture was stirred for 20 hours at room temperature and extra chloro acetic acid methyl ester (3.3 ml) was added. The mixture was stirred for another 20 hours at room temperature and again extra chloro acetic acid methyl ester (2 ml) was added. The resulting mixture was stirred for 24 hours and then the solids were filtered off and washed with DMW. Et₂O (800 ml) was added and the mixture was washed 3 times with H₂O (500 ml). The organic layer was separated, dried (MgSO₄), filtered off and the solvent was evaporated, then co-evaporated with toluene. The residual oil (23.4 g) was filtered over silica gel (eluent: CH₂Cl₂/CH₃OH 99/1). The product fractions were collected and the solvent was evaporated, finally co-evaporated with toluene. Yield: 20.6 g of intermediate 4 (99.7%).
d. Preparation of Intermediate 5

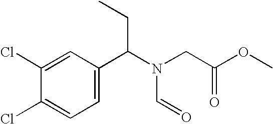

A solution of formic acid (7.5 ml) and intermediate 4 (prepared according to A1.c) (0.0746 mol) in xylene, p.a. (225 ml) was stirred and refluxed for 4 hours and then the reaction mixture was allowed to reach room temperature. The mixture was washed 2 times with H₂O (2×200 ml), with a saturated aqueous NaHCO₃ solution (200 ml) and with brine (200 ml), then the separated organic layer was dried (MgSO₄) and filtered off. Finally, the solvent was evaporated. Yield: 21.3 g of intermediate 5 (93.9%).
e-1. Preparation of Intermediate 6

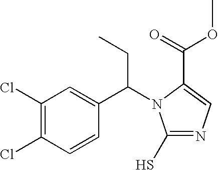

NaOCH₃ (0.07 mol) was added to a stirring solution of intermediate 5 (prepared according to A1.d) (0.066 mol) and methyl ester formic acid (0.19 mol) in THF, p.a. (100 ml) under N₂ and the reaction mixture was stirred at room temperature for 40 hours. The solvent was evaporated and the crude residue was stirred in water (90 ml). The aqueous layer was washed 2 times with Et₂O (50 ml) and CH₃OH (60 ml) was added. HCl, 36%, p.a. (15.7 ml) was added and the solution was heated on an oil-bath and stirred at 45° C. for 24 hours, then KSCN (0.11 mol) was added. The mixture was stirred at room temperature for 40 hours and at 80° C. for 5 hours. The reaction mixture was allowed to reach room temperature and the solids were filtered off, washed with H₂O/CH₃OH (2/1), then dried at 60° C. (vacuum). Yield: 19.35 g of a fraction which was re-crystallized from CH₃CN (200 ml), filtered off, washed with CH₃CN and dried at 50° C. (vacuum). Yield: 17.2 g of intermediate 6 (75.5%).
e-2. Preparation of Intermediate 44

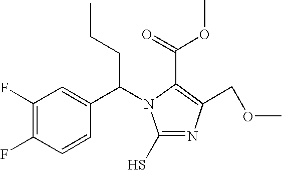

Methyl 2-[[1-(3,4-difluorophenyl)butyl](formyl)amino]acetate (prepared according to A1.d) (15.0 g, 53 mmol) dissolved in 250 ml of absolute ether was cooled at stirring to −78° C. in an argon atmosphere, and 55 ml of 2M solution of lithium diisopropylamide in THF/hexane was added dropwise at −78° C. The mixture was stirred for an additional 0.5 hour at the same temperature. Methoxyacetic acid chloroanhydride (6.9 g, 63.6 mmol) dissolved in 40 ml of dry THF was added dropwise to the mixture, and the reaction temperature was allowed to adjust to room temperature for 2 hours. A mixture of methanol (270 ml), water (135 ml), KCNS (13.43 g) and 67 ml of conc. HCl was added to the mixture. The resulting mixture was heated to 70° C. and stirred for 18 hours at this temperature. Then, it was allowed to cool to room temperature., and neutralized with an aqueous solution of NaHCO₃ and extracted with methylene chloride. The extract was dried over MgSO₄ and concentrated in-vacuo. The residue obtained was subjected to flash-chromatography on silica gel 60/100 (CH₂Cl₂). The fraction containing the desired product (according to LC/MS) was concentrated, and treated with a mixture of hexane/ether (1/1). Yield: 2.46 g (12.5%) of intermediate 44.
f-1. Preparation of Intermediate 7

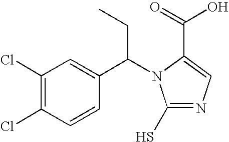

A solution of intermediate 6 (prepared according to A1.e) (0.004 mol) in NaOH (1N) (7.5 ml), methanol p.a. (10 ml) and THF, p.a. (20 ml) was stirred at room temperature for 20 hours, then stirred for 5 days at 75° C. Extra NaOH (1N) (7.5 ml) was added and the reaction mixture was stirred for 1 hour at 75° C., then the mixture was allowed to reach room temperature. H₂O (45 ml), then Et₂O (50 ml) was added and the reaction mixture was stirred for 30 minutes. The aqueous layer was separated, washed with EtOAc/Hexane (2×50 ml, 1/1) and acidified with HCl (1N) to pH 3. The mixture was extracted with CH₂Cl₂/CH₃OH (95/5), then the organic layer was separated, dried (MgSO₄), filtered and the solvent was evaporated. The residue was dissolved in Et₂O/Hexane/CH₂Cl₂ (1/1/1) and concentrated at 60° C. (without vacuum) until crystallisation started, then the mixture stood for 30 minutes. The precipitate was filtered off, washed with Hexane/Et2O (3/1) and dried (vacuum, 50° C.), followed by drying with vacuo pump for 5 hours). Yield: 0.55 g of intermediate 7 (41.5%).
f-2. Preparation of Intermediate 42

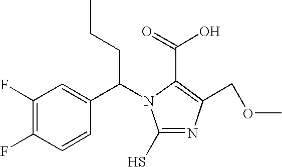

A mixture of intermediate 42 (0.0045 mol) and NaOH 1N (0.025 mol) in methanol (25 ml) was stirred for 24 hours at 60° C., then extra NaOH 1N (10 ml) was added and the reaction mixture was stirred for 20 hours at 60° C. A part of the solvent was evaporated and 1N HCl (36 ml) was added to the concentrate. The product was precipitated as an oil and was extracted with CH₂Cl₂. The organic layer was separated, dried and filtered off. The solvent was evaporated and the residue was dried Yield: 1.4 g of intermediate 42.
g. Preparation of Intermediate 8

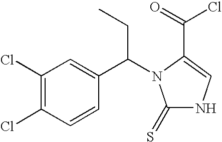

A mixture of intermediate 7 (prepared according to A1.f) (0.001 mol) in SOCl₂ (10 ml) was stirred and refluxed for 2 hours and then the solvent was evaporated. Toluene (p.a.) was added 2 times to the residue and the solvent was evaporated after each addition. Yield: 0.35 g of intermediate 8.
h. Preparation of Intermediate 9

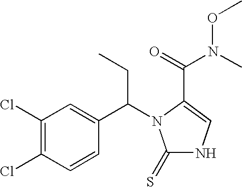

A mixture of N-methoxymethanamine hydrochloride (0.0017 mol) and Et₃N (0.005 mol) in THF (10 ml) was stirred at room temperature and a mixture of intermediate 8 (prepared according to A1.g) (0.0015 mol) in THF (5 ml) was added dropwise over 15 minutes, then the reaction mixture was stirred overnight at room temperature and the solvent was evaporated. The residue was stirred in H₂O and the mixture was extracted with CH₂Cl₂. The organic layer was separated, dried (MgSO₄), filtered off and the solvent was evaporated. The residue was dissolved in 2-propanone (15 ml, p.a.) and a stream of SO₂ was passed through the solution for 20 minutes The solvent was evaporated and the residue was filtered over silica gel (eluent: CH₂Cl₂/CH₃OH 95/5). The product fractions were collected and the solvent was evaporated Yield: 0.310 g of intermediate 9.
i. Preparation of Intermediate 10

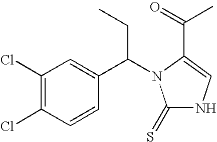

A mixture of intermediate 9 (prepared according to A1.h) (0.0008 mol) in THF (10 ml). was stirred at 0-5° C. on an ice bath and CH₃MgCl, 20% in THF (0.002 mol) was added dropwise, then the reaction mixture was stirred for 1 hour and extra CH₃MgCl, 20% in THF (0.0044 mol) was added dropwise. The mixture was stirred for 1 hour and again extra CH₃MgCl, 20% in THF (0.002 mol) was added dropwise, then the reaction mixture was stirred for 1 hour and 1N HCl (10 ml) was added dropwise under cooling with ice. H₂O was added and the mixture was extracted with CH₂Cl₂. The organic layer was separated, dried (MgSO₄), filtered off and the solvent was evaporated. The residue was dissolved in 2-propanone (p.a.) and a stream of SO₂ was passed through the solution for 20 minutes. The solvent was evaporated and the residue was purified by Flash column chromatography (eluent: EtOAc). The product fractions were collected and dissolved in CH₂Cl₂/CH₃OH (90/10), then the mixture was filtered and the filtrate's solvent was evaporated Yield: 0.060 g of intermediate 10.
i. Preparation of Intermediate 11

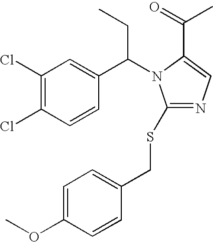

A mixture of intermediate 10 (prepared according to A1.i) (0.005 mol) in dry THF (40 ml) was stirred at room temperature and 60% NaH (0.0055 mol) was added portionwise over 10 minutes, then the mixture was stirred for 30 minutes and a mixture of 1-(chloromethyl)-4-methoxybenzene (0.0055 mol) in dry THF (10 ml) was added at once. The reaction mixture was stirred at room temperature for 6 days and the solvent was evaporated. The residue was stirred in H₂O and extracted with CH₂Cl₂. The organic layer was separated, dried (MgSO₄), filtered off and the solvent was evaporated. Yield: 2.2 g of intermediate 11.
k. Preparation of Intermediate 12

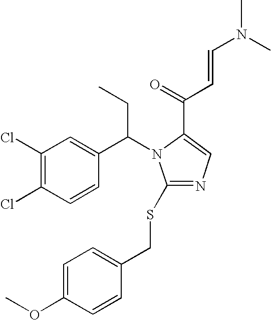

A mixture of intermediate 11 (prepared according to A1.j) (0.00115 mol) in 1,1-dimethoxy-N,N-dimethylmethanamine (10 ml) was stirred at 100° C. for 18 hours and then the solvent was evaporated. The residue was dissolved in toluene and the solvent was evaporated again. Yield: 0.580 g of intermediate 12.
1-1. Preparation of Intermediate 13 and 14

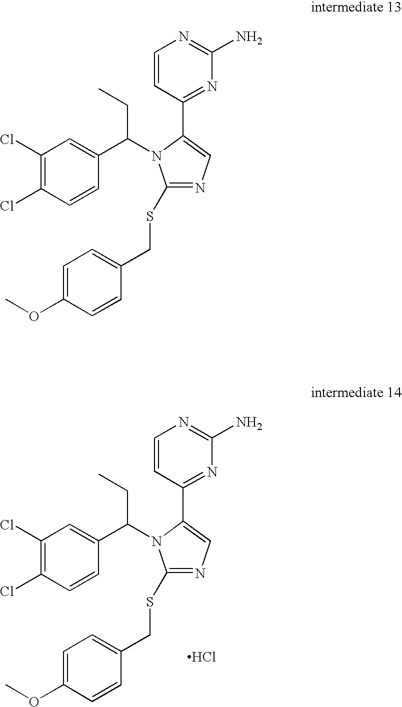

A mixture of Na (0.0008 mol) in ethanol (4 ml) was stirred under N₂ at room temperature until complete dissolution and guanidine (0.0004 mol) was added, then the mixture was stirred for 30 minutes and a mixture of intermediate 12 (prepared according to A1.k) (0.00055 mol) in ethanol (1 ml) was added. The reaction mixture was stirred in a closed vessel at 100° C. for 20 hours and evaporated. The crude compound was purified over SiO₂ (eluent: CH₂Cl₂/CH₃OH 98/2) and the solvent was evaporated, yielding intermediate 13. The residue was dissolved in 2-propanol and converted into the hydrochloric acid salt (1:1) with HCl/2-propanol. The crystallised salt was filtered off, washed with DIPE and dried. Yield: 0.320 g of intermediate 14.
1-2. Preparation of Intermediate 15

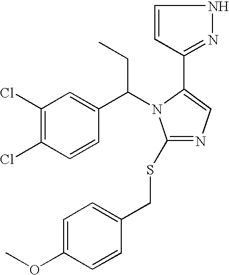

A mixture of intermediate 12 (prepared according to A1.k) (0.00115 mol) and hydrazine monohydrate (0.007 mol) in ethanol (20 ml) was stirred and refluxed on an oil bath for 3 hours. The solvent was evaporated and the residue was filtered over silica gel (eluent: CH₂Cl₂/CH₃OH 98/2). The product fractions were collected and the solvent was evaporated at 50° C. under a stream of N₂. Yield: 0.240 g of intermediate 15.

### EXAMPLE A2

a. Preparation of Intermediate 16

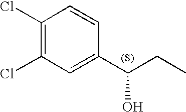

A mixture of N,N′-(1R,2R)-1,2-cyclohexanediylbis[1,1,1-trifluoromethanesulfonamide] (0.005 mol) and Ti(i-PrO)₄ (0.030 mol) in toluene (q.s.) was degassed and placed under Ar-flow, then the reaction mixture was stirred for 20 minutes at 40° C. and cooled to −78° C. Et₂Zn (0.030 mol) was added dropwise and after 20 minutes, a mixture of 3,4-dichlorobenzaldehyde (0.0250 mol) in toluene (q.s.) was added dropwise. The reaction mixture was allowed to reach 0° C. The mixture was stirred overnight at room temperature, then quenched with HCl (2N). This mixture was extracted with CH₂Cl₂. The separated organic layer was washed, dried, filtered and the solvent evaporated. The residue was purified by column chromatography over silica gel (eluent CH₂Cl₂ /CH₃OH 98/2). The product fractions were collected and the solvent was evaporated. Yield: 5.1 g of intermediate 16 .

The R isomer can be prepared by the above reaction by using N,N′-(1S,2S)-1,2-cyclohexanediylbis[1,1,1-trifluoromethanesulfonamide] as catalyst.
b. Preparation of Intermediate 17

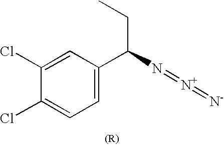

A mixture of intermediate 16 (prepared according to A2.a) (0.025 mol) and diphenylphosphoryl azide (0.030 mol) in toluene (50 ml) was stirred at 0° C. and 2,3,4,6,7,8,9,10-octahydropyrimido[1,2-a]azepine (0.030 mol) was added. The reaction mixture was stirred for 2 hours at 0° C., then stirred overnight at room temperature. The mixture was diluted with water and toluene. The organic layer was separated, washed once with water, once with 5% HCl, and the solvent was evaporated, yielding intermediate 17, used in next reaction step.
c. Preparation of Intermediate 18

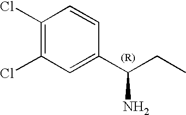

A mixture of intermediate 17 (prepared according to A2.b) (0.025 mol), triphenylphosphine (0.027 mol) in THF (70 ml) and H₂O (20 ml) was stirred overnight at room temperature. The solvent was evaporated. The residue was treated with 10% HCl. The acidic layer was washed with DIPE, then alkalized, followed by an extraction with CH₂Cl₂. The separated organic layer was dried, filtered and the solvent evaporated. The residue was purified by column chromatography over silica gel. The product fractions were collected and the solvent was evaporated. Yield: 1.1 g of intermediate 18.
d. Preparation of Intermediate 19

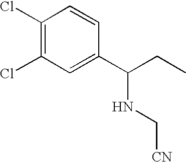

A solution of intermediate 3 (prepared according to A1.b) (0.0116 mol) in Et₃N (0.013 mol) and DMF, p.a., dried on molecular sieves (20 ml) was stirred on an ice bath. A solution of chloroacetonitrile (0.0128 mol) in DMF, p.a., dried on molecular sieves, (2.5 ml) was added dropwise. The reaction mixture was stirred at room temperature for 6 hours. More chloroacetonitrile (0.0063 mol) in DMF, p.a., dried on molecular sieves (1 ml) was added dropwise. The reaction mixture was stirred for another 24 hours. More chloroacetonitrile (0.0063 mol) in DMF, p.a., dried on molecular sieves (1 ml) was added dropwise and the reaction mixture was stirred for another 24 hours. More Et₃N (1 ml) was added, then more chloroacetonitrile(0.0079 mol) in DMF, p.a., dried on molecular sieves, (1 ml) was added dropwise. The reaction mixture was stirred for 20 hours. The precipitate was filtered off. The filtrate was poured out into Et₂O (200 ml) and washed with H₂O/NaHCO₃ (10%; 100 ml) and H₂O (2×). The separated organic layer was dried (MgSO₄), filtered and the solvent was evaporated and co-evaporated with toluene. The residue was purified over silica gel (eluent: CH₂Cl₂/MeOH 99:1). The desired fractions were collected and the solvent was evaporated and co-evaporated with toluene. Yield: 2.3 g of intermediate 19 (81.6%).
e. Preparation of Intermediate 20

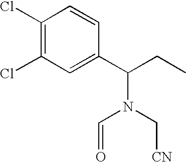

A mixture of intermediate 19 (prepared according to A2.d) (0.021 mol) in n-butyl formate (25 ml) was stirred and refluxed for 48 hours and then cooled. The solvent was evaporated and the residue was diluted with CH₂Cl₂. The resulting mixture was washed with water, dried and the solvent was evaporated. Yield: 5.25 g of intermediate 20.
f Preparation of Intermediate 21

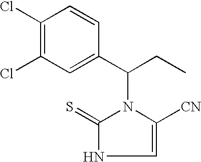

NaOMe (prepared in situ) (q.s.) was added dropwise to a mixture of intermediate 20 (prepared according to A2.e) (0.022 mol) and acetaldehyde (0.022 mol) in THF (100 ml). After 7 hours, the solvent was evaporated and the residue was partitioned between water and ether. The aqueous layer was acidified with concentrated HCl to pH 2-3 and extracted with CH₂Cl₂. The extract was evaporated and the residue was dissolved in CH₃OH/H₂O. KSCN (0.050 mol) was added, followed by concentrated HCl (5 ml) and then the reaction mixture was stirred and refluxed over the weekend. The mixture was cooled and the solvent was evaporated. The residue was partitioned between water and CH₂Cl₂, then the organic layer was separated, dried and the solvent was evaporated. The residue was purified by flash column chromatography over silica gel (eluent: Hexane/EtOAc 80/20). The product fractions were collected and the solvent was evaporated. Yield: 2 g of intermediate 21. Remark: NaOMe was generated in situ with NaH and CH₃OH in THF.
g. Preparation of Intermediate 22

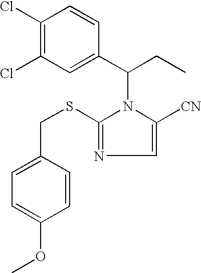

A mixture of intermediate 21 (prepared according to A2.f) (0.0128 mol) in trifluoroacetic acid (5 ml) and CH₂Cl₂ (40 ml) was stirred at 0-5° C. on an ice bath and a solution of 4-methoxybenzenemethanol (0.017 mol) in CH₂Cl₂ (10 ml) was added dropwise over 30 minutes at 0-5° C., then the reaction mixture was stirred for 2 hours at 0-5° C. and the solvent was evaporated (vacuum). The residue was dissolved in CH₂Cl₂ and washed with H₂O and with NaHCO₃. The organic layer was separated, dried (MgSO₄), filtered off and the solvent was evaporated. The residue was purified over silica gel (eluent: CH₂Cl₂/CH₃OH 99/1). The product fractions were collected and the solvent was evaporated. Yield: 4.3 g of intermediate 22.
h. Preparation of Intermediate 23

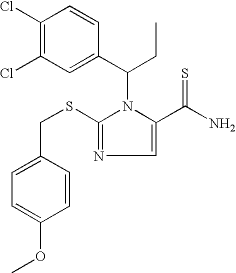

A mixture of intermediate 22 (prepared according to A2.g) (0.01 mol) and N-ethyl-N-(1-methylethyl)-2-propanamine (0.02 mol) in pyridine (100 ml) was stirred at 80° C. and then H₂S (gas) was passed through the solution for 5 hours, then N₂ was passed through for 2 hours at 80° C. in order to remove the H₂S. The solvent was evaporated and the residue was dissolved in CH₂Cl₂. The solution was washed with H₂O and with 1N HCl. The organic layer was separated, dried (MgSO₄), filtered off and the solvent was evaporated. The residue was purified over silica gel (eluent: CH₂Cl₂/CH₃OH 99/1). The product fractions were collected and the solvent was evaporated. Yield: 3 g of intermediate 23.
i-1. Preparation of Intermediate 24

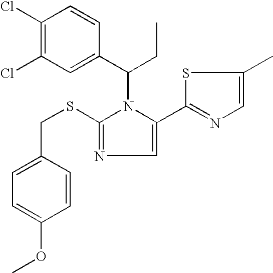

A mixture of intermediate 23 (prepared according to A2.h) (0.0005 mol) and 1-chloro-2-propanone (0.00055 mol) in ethanol (4 ml) was stirred at 80° C. for 3 hours, then the mixture was left to stand overnight at room temperature and the solvent was evaporated. The residue was purified over silica gel (eluent: CH₂Cl₂/CH₃OH 97/3). The product fractions were collected and the solvent was evaporated, giving fraction (I). The fractions containing intermediate 23 were collected and the solvent was evaporated. Ethanol (4 ml) and extra 1-chloro-2-propanone (0.04 ml) were added to the residue and the reaction mixture was stirred for 3 hours. The solvent was evaporated and the residue was filtered over silica gel (eluent: CH₂Cl₂/CH₃OH 97/3). The product fractions were collected and the solvent was evaporated, giving fraction (II). Finally, fraction (I) and fraction (II) were combined. Yield: 0.030 g of intermediate 24
i-2. Preparation of Intermediate 25

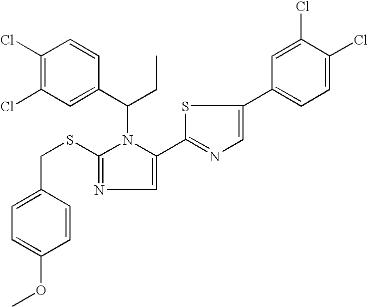

A mixture of intermediate 23 (prepared according to A2.h) (0.0005 mol) and 2-bromo-1-(3,4-dichlorophenyl)ethanone (0.0007 mol) in ethanol (4 ml) was stirred in a closed vessel for 18 hours at room temperature and then the solvent was evaporated. The residue was filtered 2 times (eluent: CH₂Cl₂/CH₃OH 99/1, 100/0), then the product fractions were collected and the solvent was evaporated. Yield: 0.120 g of intermediate 25.

### EXAMPLE A3

a. Preparation of Intermediate 26

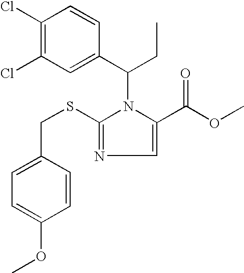

A mixture of intermediate 6 (prepared according to A1.e) (0.03 mol) in trifluoroacetic acid (20 ml) and CH₂Cl₂ (150 ml) was stirred at 0-5° C. on an ice bath and a mixture of 4-methoxybenzenemethanol (0.033 mol) in CH₂Cl₂ (50 ml) was added dropwise over 30 minutes at 0-5° C., then the reaction mixture was stirred for 1 hour at 0-5° C. and the solvent was evaporated. The residue was dissolved in CH₂Cl₂ and washed with H₂O and with NaHCO₃. The organic layer was separated, dried (MgSO₄), filtered off and the solvent was evaporated, finally the residue was dried. Yield: 13.9 g of intermediate 26.
b. Preparation of Intermediate 27

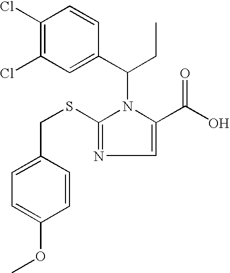

A mixture of intermediate 26 (prepared according to A3.a) (0.03 mol) in 1N NaOH (150 ml) and methanol (150 ml) was stirred at 80° C. for 18 hours and then the organic solvent (methanol) was evaporated. 1N HCl (1501ml) was added to the aqueous concentrate, then the resulting precipitate was filtered off and dried. Yield: 12 g of intermediate 27.
c. Preparation of Intermediate 28

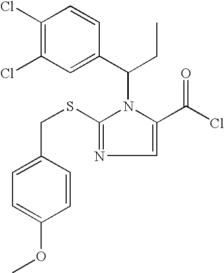

A mixture of intermediate 27 (prepared according to A3.b) (0.026 mol) in SOCl₂ (100 ml) was stirred and refluxed for 2 hours, then the solvent was evaporated and co-evaporated 2 times with toluene. Yield: 12.5 g of intermediate 28.
d. Preparation of Intermediate 29

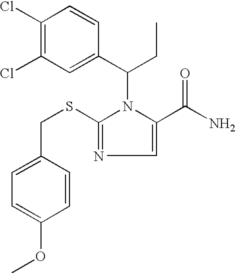

A solution of intermediate 28 (prepared according to A3.c) (0.026 mol) in THF (200 ml) was added dropwise to stirring NH₃/H₂O (50 ml) at room temperature and the reaction mixture was stirred for 2 hours. The solvent was partly (1/2) evaporated and the concentrate was extracted with CH₂Cl₂. The organic layer was separated, dried (MgSO₄), filtered off and the solvent was evaporated. The residue was purified over silica gel (eluent: CH₂Cl₂/CH₃OH 95/5) The product fractions were collected and the solvent was evaporated. Yield: 3.5 g of intermediate 29.
e. Preparation of Intermediate 30

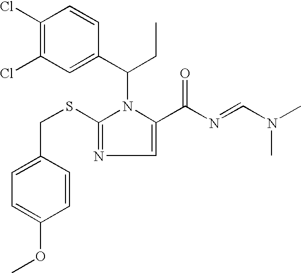

A mixture of intermediate 29 (prepared according to A3.d) (0.0022 mol) in DMF/DMA (25 ml) was stirred at 100° C. for 18 hours and then the solvent was evaporated. The residue was filtered 2 times over silica gel (eluent: CH₂Cl₂/CH₃OH 99/1), then the product fractions were collected and the solvent was evaporated. Yield: 0.51 g of intermediate 30.
f. Preparation of Intermediate 31

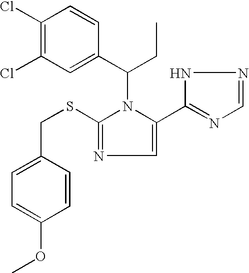

A mixture of intermediate 30 (prepared according to A3.e) (0.001 mol) and hydrazine monohydrate (0.0015 mol) in acetic acid (5 ml) was stirred in a closed vessel for 2 hours at 90° C. and then the solvent was evaporated. The residue was dissolved in CH₂Cl₂ and washed with H₂O and with K₂CO₃. The organic layer was separated, dried (MgSO₄), filtered off and the solvent was evaporated. The residue was purified by column chromatography over silica gel (eluent: CH₂Cl₂/CH₃OH 97/3). The product fractions were collected and the solvent was evaporated. Yield: 0.200 g of intermediate 31.

### EXAMPLE A4

a. Preparation of Intermediate 32

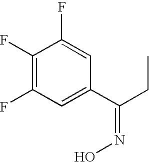

A mixture of 1-(3,4,5-trifluorophenyl)-1-propanone (0.106 mol), hydroxylamine (0.150 mol) and NaOAc (0.150 mol) in methanol (q.s.) was stirred overnight at room temperature, then the reaction mixture was filtered and the filtrate was evaporated. Yield: 21.5 g of intermediate 32.
b. Preparation of Intermediate 33

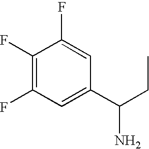

A mixture of intermediate 32 (prepared according to A4.a) (0.11 mol) in CH₃OH/NH₃ (500 ml) was hydrogenated with Raney Nickel (2 g) as a catalyst. After uptake of H₂ (2 equivalents), the catalyst was filtered off and the filtrate was evaporated. Yield: 20 g of intermediate 33.
c. Preparation of Intermediate 34

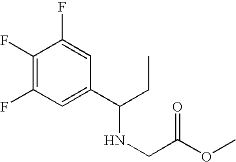

A mixture of intermediate 33 (prepared according to A4.b) (0.105 mol) and bromoacetic acid methyl ester (0.196 mol) in Et₃N (30 ml) was reacted overnight at room temperature and then extra bromoacetic acid methyl ester (10 g) was added. N,N-dimethyl-4-pyridinamine (1 g) was added and the reaction mixture was stirred for 3 days at room temperature. The solvent was evaporated and the residue was diluted with CH₂Cl₂. The resulting solids were filtered off and dried (vacuum) at 50° C. Yield: 6.8 g of intermediate 34.
d. Preparation of Intermediate 35

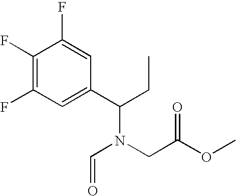

A mixture of acetylchloride (0.1 mol) and HCO₂Na (10 g) in Et₂O (100 ml) was stirred for 6 hours at room temperature and then the solids were filtered off. A mixture of intermediate 34 (prepared according to A4.c) (0.024 mol) in Et₂O (q.s.) was added and the reaction mixture was stirred for 4 hours at room temperature. The solvent was evaporated and the residue was quenched with NaHCO₃, then extracted with CH₂Cl₂. The organic layer was separated, dried (MgSO₄), filtered off and the solvent was evaporated. Yield: 4.4 g of intermediate 35.
e. Preparation of intermediate 36

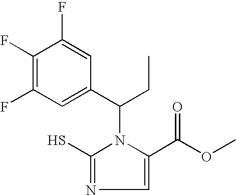

t-BuONa (2 g) was added to a mixture of intermediate 35 (prepared according to A4.d) (0.0052 mol) in acetic acid methyl ester (3 ml) and THF (80 ml) at room temperature and the mixture was reacted for 20 minutes, then water (20 ml) was added and the solvent was concentrated until a volume of 30 ml was left. Water (20 ml) and methanol (40 ml) were added to the residue, followed by the addition of KSCN (3 g) and HCl (3 ml). The reaction mixture was heated for 3 hours and quenched with Na₂CO₃ (to neutral). The solvent was concentrated until a volume of 40 ml was left and the concentrate was extracted with CH₂Cl_{2,}. The organic layer was dried over MgSO₄, filtered and evaporated. Yield: 1.5 g of intermediate 36 (88%).
f. Preparation of Intermediate 37

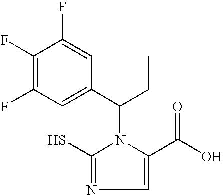

A mixture of intermediate 36 (prepared according to A4.e) (0.0015 mol) in 1M NaOH (5 ml) and methanol (3 ml) was stirred and refluxed for 24 hours at 100° C., then the reaction mixture was cooled and the solvent was evaporated. The residue was quenched with concentrated HCl and the desired product was collected. Yield: 360 mg of intermediate 37.

### EXAMPLE A5

a. Preparation of Intermediate 38

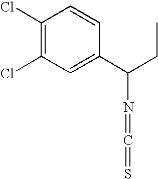

N-ethyl-N-(1-methylethyl)-2-propanamine (0.1 mol) was added to a stirring mixture of intermediate 3 (prepared according to A1.b) (0.0415 mol) in CH₂Cl₂, p.a. (100 ml) under N₂. After 15 minutes of stirang, the reaction mixture was put on an ice bath and a solution of carbonothioic dichloride (0.0457 mol) in CH₂Cl₂, p.a. (15 ml) was added dropwise at 0° C. The mixture was stirred at 0° C. for 30 minutes and at room temperature for 18 hours, then extra N-ethyl-N-(1-methylethyl)-2-propanamine (9 ml) was added and the resulting mixture was stirred for 2 hours. The mixture was washed 2 times with H₂O, once with HCl (1N) and again with H₂O. The organic layer was separated, dried (MgSO₄), filtered off and the solvent was evaporated, then co-evaporated with toluene. The residue was purified by column chromatography over silica gel (eluent: CH₂Cl₂/Hexane 15/85). The product fractions were collected and the solvent was evaporated. Yield: 7.4 g of intermediate 38 (72.4%).
b-1. Preparation of Intermediate 39

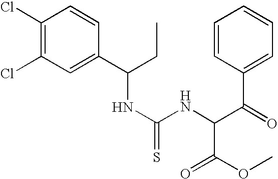

Beta-oxo-phenylalanine methyl ester monohydrochloride (0.00175 mol), followed by K₂CO₃ (0.00175 mol) and then H₂O (5 ml) were added to a solution of intermediate 38 (prepared according to A5.a) (0.00175 mol) in THF (20 ml) and the reaction mixture was stirred at room temperature for 18 hours. The mixture was poured out into H₂O (50 ml) and extracted with CH₂Cl₂. The organic layer was separated, dried (MgSO₄), filtered off and the solvent was evaporated. The residue was purified by flash column chromatography (eluent: CH₂Cl₂/CH₃OH). The product fractions were collected and the solvent was evaporated. Yield: 0.095 g of intermediate 39 (12.4%).
b-2. Preparation of Intermediate 40

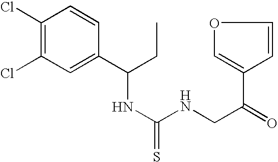

2-amino-1-(2-furanyl)-ethanone monohydrochloride (0.00146 mol) was added to a solution of intermediate 38 (prepared according to A5.a) (0.00122 mol) in 1,4-dioxane, p.a. (6 ml), then H₂O (0.5 ml) was added followed by K₂CO₃ (0.00122 mol). The reaction mixture was stirred at room temperature for 18 hours and the solvent was evaporated, yielding intermediate 40. The residue was used further in the next step.

### EXAMPLE A6

Preparation of Intermediate 41

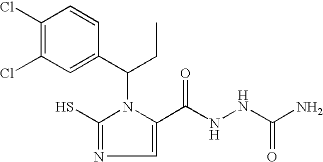

Hydrazinecarboxamide monohydrochloride (0.0078 mol) was added to a solution of intermediate 8 (prepared according to A1.g) (0.0052 mol) in THF, p.a. dried on molecular sieves (40 ml) under N₂, then N-ethyl-N-(1-methylethyl)-2-propanamine (0.016 mol) was added and the reaction mixture was stirred for 18 hours at room temperature. The solvent was evaporated and the residue was stirred in a half saturated aqueous K₂CO₃ solution. The resulting mixue was washed with CH₂Cl₂/CH₃OH (95/5) and acidified with concentrated HCl. The formed precipitate was filtered off, washed with H₂O and with DIPE and then dried (vacuum) at 50° C. Yield: 0.42 g of intermediate 41.

### EXAMPLE A7

Preparation of Intermediate 43

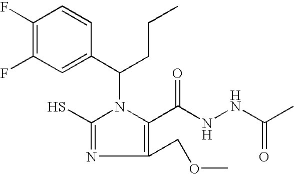

A mixture of intermediate 42 (prepared according to A1.f-2) (0.001 mol), N′-(ethylcarbonimidoyl)-N,N-dimethyl-1,3-propanediamine (0.001 mol) and 1-hydroxy-1H-benzotriazole (0.001 mol) in DMF (5 ml) was stirred for 30 minutes at room temperature, then hydrazide acetic acid (0.003 mol) was added and the reaction mixture was stirred overnight at room temperature. The solvent was evaporated, then the residue was stirred in H₂O and the mixture was extracted with CH₂Cl₂/CH₃OH (90/10). The organic layer was separated, dried, filtered off and the solvent was evaporated. The obtained residue was stirred in CH₂Cl₂ and after solidification the desired product was filtered off and dried. Yield: 0.170 g of intermediate 43.

B. Preparation of the Final Compounds

### EXAMPLE B1

Preparation of Compound 1

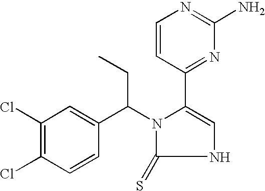

A mixture of intermediate 14 (prepared according to A1.1-1) (0.00055 mol) in trifluoroacetic acid (3 ml) was stirred at 80° C. in a closed vessel for 4 hours and then the solvent was evaporated at 80° C. under a stream of N₂. The residue was stirred in CH₂Cl₂ and treated with H₂₀ and K₂CO₃. The organic layer was separated, dried (MgSO₄), filtered off and the solvent was evaporated. The residue was stirred in EtOAc (2 ml) and after filtration the desired product was dried. Yield: 0.020 g of compound 1.

### EXAMPLE B2

Preparation of Compound 2

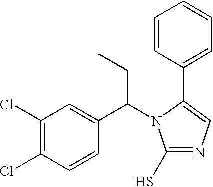

2-amino-2-benzoylacetamide monohydrochloride (0.000158 mol), H₂O (0.5 ml) and K₂CO₃ (0.000154 mol) were added to a solution of intermediate 38 (prepared according to A5.a) (0.000154 mol) in 1,4-dioxane, p.a. (2.5 ml) and then the reaction mixture was stirred vigorously for 20 hours. 36% HCl, p.a. (0.5 ml) was added and the resulting mixture was stirred for 4 hours at 75° C. and then allowed to reach room temperature, H₂O was added and the mixture was extracted with EtOAc. The organic layer was separated, dried (MgSO₄), filtered off and the solvent was evaporated. The residue was purified by reversed phase high-performance liquid chromatography. The desired product fractions were collected and the organic volatiles were evaporated. The product was extracted with CH₂Cl₂ and the separated organic layer was evaporated. Yield: 0.0076 g of compound 2.

### EXAMPLE B3

a. Preparation of Compound 3

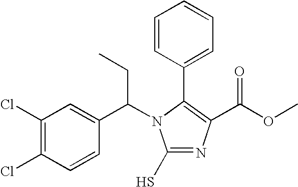

A solution of intermediate 39 (prepared according to A5.b-1) (0.0002 mol) in acetic acid (6 ml) was stirred for 18 hours in a sealed tube at 100° C., then the reaction mixture was allowed to reach room temperature and was poured out into H₂O. CH₂Cl₂ was added, then a saturated K₂CO₃ solution was added until a clear biphasic solution was formed. The organic layer was separated, dried (MgSO₄), filtered off and the solvent was evaporated, then co-evaporated with toluene. The residue was purified by high-performance liquid chromatography over RP-18 (eluent: (10% NH₄OAc in H₂O)/CH₃OH/CH₃CN). The product fractions were collected and the solvent was evaporated for 50%. The concentrate was extracted with CH₂Cl₂ and the separated organic layer was evaporated. Yield: 0.011 g of compound 3.
b. Preparation of Compound 4

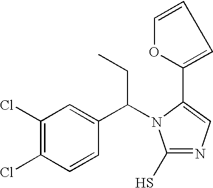

36% HCl, p.a. (2 ml) was added to a solution of intermediate 40 (prepared according to A5.b-2) (0.0012 mol) in 1,4-dioxane, p.a. (12 ml) and the resulting solution was stirred at 65° C. for 18 hours. H₂O (10 ml) was added and the mixture was extracted with EtOAc. The organic layer was separated, dried (MgSO₄), filtered off and the solvent was evaporated. The residue was dissolved in 2-propanone (15 ml, p.a.) and the solution was treated with SO2 (gas) for 15 minutes. The solvent was evaporated and the residue was purified by flash column chromatography (gradient eluent: CH₂Cl₂/CH₃OH 99.8/0.2->99.6/0.4). The product fractions were collected and the solvent was evaporated. Yield: 0.0134 g of compound4.

### EXAMPLE B4

Preparation of Compound 5

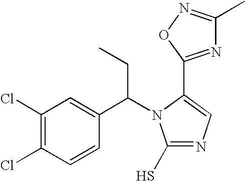

A mixture of intermediate 6 prepared according to A1.e) (0.00072 mol) and N-hydroxy-ethanimidamide (0.00181 mol) in methanol (3 ml) and NaOCH₃/CH₃OH (30%) (1 ml) was reacted at 100° C. in a sealed tube for 18 hours and then the reaction mixture was quenched with an aqueous NH₄Cl solution. The solvent was evaporated and the residue was extracted with CH₂Cl₂. The organic layer was separated, filtered through a drying cartridge and the solvent was evaporated. The residue was purified by high-performance liquid chromatography. The product fractions were collected and the solvent was evaporated. Yield: 0.110 g of compound 5.

### EXAMPLE B5

Preparation of Compound 6

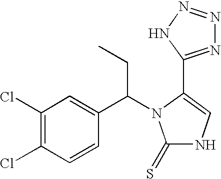

tBuOK (10 g) was added dropwise to a mixture of intermediate 20 (prepared according to A2.e) (0.063 mol) and methyl ester formic acid (0.32 mol) in THF (300 ml). After 7 hours, the solvent was evaporated and the residue was partitioned between water and ether. The aqueous layer was acidified with concentrated HCl to pH 2-3 and extracted with CH₂Cl₂. The extract was evaporated and the residue was dissolved in CH₃OH/H₂O. KSCN (15 g) was added, followed by concentrated HCl (q.s.) and then the reaction mixture was stirred and refluxed over the weekend. The mixture was cooled and the solvent was evaporated. The residue was partitioned between water and CH₂Cl₂, then the organic layer was separated, dried and the solvent was evaporated. The residue was purified by flash column chromatography over silica gel (eluent: Hexane/EtOAc 80/20). The product fractions were collected and the solvent was evaporated. Then the product fractions were collected and the solvent was evaporated, yielding 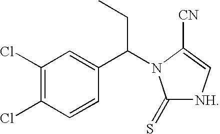
A mixture of 0.200 g of said intermediate, 0.5 g of Bu₃SnN₃ in 0.5 ml toluene was heated overnight at 130° C. and the mixture was purified by reversed phase high performance liquid chromatography resulting in 20 mg of compound 6.

### EXAMPLE B6

a. Preparation of Compound 7

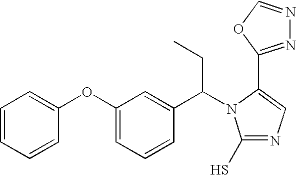

A mixture of intermediate 44 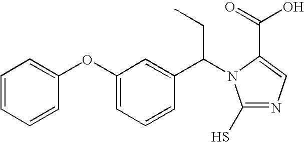
(prepared according to A1.f-1) (0.00037 mol) in SOCl₂ (25 ml) was stirred and refluxed for 4 hours, then the reaction mixture was cooled and the solvent was evaporated. The oily residue was diluted with THF (10 ml) and treated with hydrazinecarboxaldehyde (0.0042 mol), then the mixture was stirred for 30 minutes at room temperature and the solvent was evaporated. The residue was diluted with CH₂Cl₂ and dried by Solid Phase Extraction. The solvent was evaporated and the residue was diluted with CH₃CN. The resulting mixture was treated with POCl₃ (10% w) (1 ml) in CH₃CN (5 ml) and heated for 5 hours. The mixture was cooled and the solvent was evaporated. The residue was extracted with CH₂Cl₂ and dried by Solid Phase Extraction, then the solvent was evaporated and the residue was purified by Reversed Phase high-performance liquid chromatography. The product fractions were collected and the solvent was evaporated. Yield: 22 mg of compound 7.
b. Preparation of Compound 8

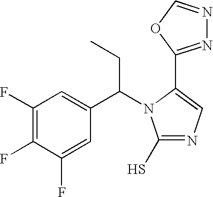

A mixture of intermediate 37 (prepared according to A4.f) (0.001 mol) in SOCl₂ (5 ml) was heated at 70° C. for 4 hours, then the mixture was cooled and the solvent was evaporated. A part (1/3) of this fraction was taken up in THF (4 ml) and treated with hydrazinecarboxaldehyde (0.0025 mol), then the reaction mixture was heated for 1 hour. The solvent was evaporated and the residue was taken up in water (0.5 ml) and CH₂Cl₂. The resulting mixture was extracted with a Solid Phase Extraction cartridge and the solvent was evaporated. The residue was taken up in CH₃CN (5 ml), then the mixture was treated with a 1M POCl₃ solution (0.0004 mol) and heated at 80° C. for 12 hours. The resulting mixture was cooled and quenched with an aqueous NaHCO₃ solution. The solvent was evaporated and the residue was purified by Reversed Phase high-performance liquid chromatography. Finally, the product fractions were collected and the solvent was evaporated. Yield: 20 mg of compound 8.

### EXAMPLE B7

Preparation of Compound 9

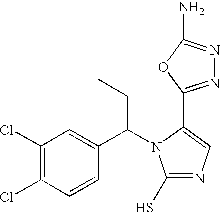

A mixture of intermediate 41 (prepared according to A6) (0.0009 mol) in CH₃CN (15 ml) was stirred on a hot oil bath and phosphorus oxychloride (0.00107 mol) was added at 50° C. and then the reaction mixture was stirred further for 2 hours at 90° C. The mixture was allowed to reach room temperature and was poured out into a half saturated, aqueous NaHCO₃ solution and extracted with CH₂Cl₂/CH₃OH 98/2. The organic layer was separated, dried (MgSO₄), filtered off and the solvent was evaporated. The residue (0.21 g) was purified by reversed phase high-performance liquid chromatography (NH₄HCO₃-buffer). The product fractions were collected and the solvent was evaporated. Yield: 0.069 g of compound 9.

### EXAMPLE B8

Preparation of Compound 10

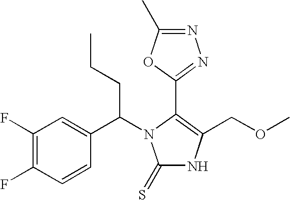

A mixture of intermediate 43 (prepared according to A7) (0.0004 mol) and Burgess'reagent (0.0012 mol) in THF (5 ml) was stirred for 3 hours at 60° C. CH₂Cl₂ was added and the reaction mixture was washed with H₂O. The organic layer was separated, dried, filtered off and the solvent was evaporated. The residue was filtered over silica gel (eluent: CH₂Cl₂/CH₃OH 98/2). The pure product fractions were collected and the solvent was evaporated. The obtained residue was stirred in DIPE, then the desired product was filtered off and dried. Yield: 0.044 g of compound 10 (m.p.: 97.8-97.9° C.).

Tables 1 and 2 list the compounds of formula (I) which were prepared according to one of the above samples (Ex. No.)

TABLE 1

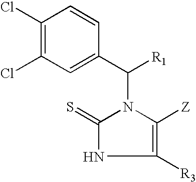

| Comp. | Exp. | | | | |
|---|---|---|---|---|---|
| No. | No. | R₁ | R₃ | Z | Properties |
| 11 | B1 | —CH₂CH₃ | H | 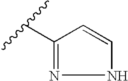 | |
| 12 | B1 | —CH₂CH₃ | H | 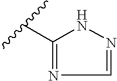 | |
| 6 | B5 | —CH₂CH₃ | H | 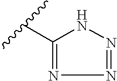 | |
| 4 | B3.b | —CH₂CH₃ | H | 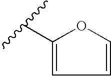 | |
| 13 | B7 | —CH₂CH₃ | H | 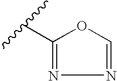 | |
| 5 | B4 | —CH₂CH₃ | H | 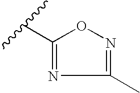 | |
| 14 | B7 | —CH₂CH₃ | H | 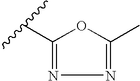 | m. p. 227-228.5° C. |
| 9 | B7 | —CH₂CH₃ | H | 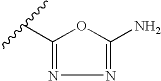 | |
| 15 | B1 | —CH₂CH₃ | H | 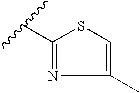 | |
| 16 | B1 | —CH₂CH₃ | H | 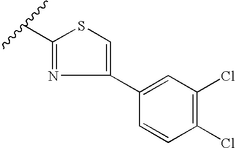 | |
| 2 | B2 | —CH₂CH₃ | H | 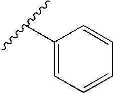 | |
| 17 | B3.b | —CH₂CH₃ | H | 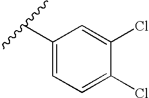 | |
| 18 | B3.b | —CH₂CH₃ | H | 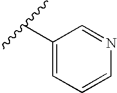 | |
| 19 | | —CH₂CH₃ | H | 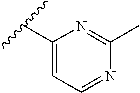 | |
| 1 | B1 | —CH₂CH₃ | H | 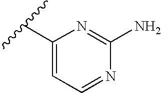 | m. p. 230.2-235.5° C. |
| 20 | B1 | —CH₂CH₃ | H | 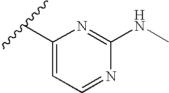 | |
| 21 | B1 | —CH₂CH₃ | H | 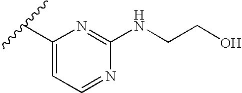 | |
| 22 | B1 | —CH₂CH₃ | H | 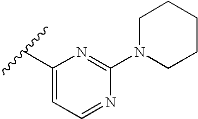 | |
| 23 | B1 | —CH₂CH₃ | H | 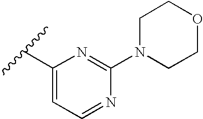 | |
| 24 | B1 | —CH₂CH₃ | H | 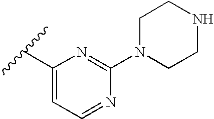 | |
| 25 | B1 | —CH₂CH₃ | H | 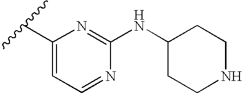 | |
| 3 | B3.a | —CH₂CH₃ | 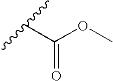 | 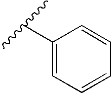 | |

TABLE 2

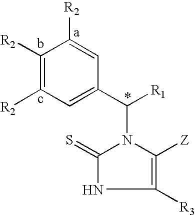

| Comp | Exp | | | R2 | | | | |
|---|---|---|---|---|---|---|---|---|
| No. | No. | R1 | a | b | c | R3 | Z | Properties |
| 26 | B1 | —CH₂CH₃ | F | —CF₃ | H | H | 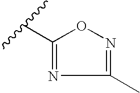 | |
| 27 | B7 | —CH₂CH₃ | F | H | F | H | 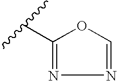 | |
| 28 | B7 | —CH₂CH₃ | F | H | F | H | 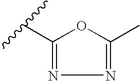 | |
| 8 | B6.b | —CH₂CH₃ | F | F | F | H | 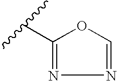 | |
| 7 | B6.a | —CH₂CH₃ | H | H | 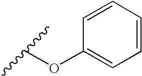 | H | 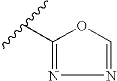 | |
| 10 | B8 | —CH₂CH₂CH₃ | F | F | H | —CH₂—OCH₃ | 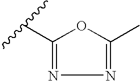 | m. p. 97.8-97.9 |
| 29 | B6.a | —CH₂CH₂CH₃ | F | F | H | H | 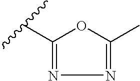 | |

C. Analytical Part
LCMS Conditions 1

The HPLC gradient was supplied by a Waters Alliance HT 2790 system with a columnheater set at 40° C. Flow from the column was split to a Waters 996 photodiode array (PDA) detector and a Waters-Micromass ZQ mass spectrometer with an electrospray ionization source operated in positive and negative ionization mode. Reversed phase HPLC was carried out on a Xterra MS C18 column (3.5 μm, 4.6×100 mm) (12 minutes column) with a flow rate of 1.6 ml/minutes. Three mobile phases (mobile phase A: 95% 25 mM ammoniumacetate±5% acetonitrile; mobile phase B: acetonitrile; mobile phase C: methanol) were employed to run a gradient condition from 100% A to 50% B and 50% C in 6.5 minutes, to 100% B in 1 minute, 100% B for 1 minute and reequilibrate with 100% A for 1.5 minute. An injection volume of 10 μL was used.

Mass spectra were acquired by scanning from 100 to 1000 in is using a dwell time of 0.1 s. The capillary needle voltage was 3 kV and the source temperature was maintained at 140° C. Nitrogen was used as the nebulizer gas. Cone voltage was 10 V for positive ionzation mode and 20 V for negative ionization mode. Data acquisition was performed with a Waters-Micromass MassLynx-Openlynx data systeni

TABLE 3
LCMS parent peak ([M⁺] defines the mass
of the compound) and retention time (minutes)

| Compound | | Retention |
|---|---|---|
| no. | [MH+] | time |
| 5 | 368 | 6.11 |
| 26 | 387 | 5.53 |
| 14 | 369 | 5.40 |
| 13 | 355 | 5.31 |
| 28 | 337 | 4.49 |
| 27 | 323 | 4.37 |
| 3 | 421 | 6.06 |
| 11 | 353 | 5.10 |
| 7 | 377 | 6.12 |
| 17 | 433 | 6.58 |
| 4 | 353 | 5.99 |
| 18 | 364 | 5.74 |
| 1 | 380 | 4.97 |
| 2 | 363 | 5.98 |
| 16 | 516 | 6.75 |
| 12 | 354 | 4.78 |
| 15 | 384 | 6.50 |
| 8 | 341 | 4.90 |
| 22 | 448 | 6.73 |
| 20 | 394 | 5.47 |
| 25 | 463 | 4.47 |
| 19 | 379 | 5.72 |
| 23 | 450 | 5.84 |
| 24 | 449 | 4.50 |
| 21 | 424 | 4.86 |
| 9 | 370 | 3.90 |

* [M⁺] defines the mass of the compound

D. Pharmacological Example
Inhibition of MCP-1 Induced Ca-Flux in Human THP-1 Cells

MCP-1 binding to the CCR₂ receptor induces a rapid and transient intracellular release of Ca²⁺ (secondary messenger) in several cell lines (Charo et al, PNAS 1994). Free Ca²⁺ levels can be measured using a Ca²⁺ sensitive dye. When the CCR₂ receptor is blocked with a CCR₂ receptor antagonist, the MCP-1 induced release of Ca²⁺ is inhibited.

Human THP-1 cells (monocytic cell line, ATCC TIB-202) were cultured in RPMI 1640 medium supplemented with 10% fetal calf serum (FCS), 1% L-Glutamine, penicillin (50 U/ml) and streptomycin (50 μg/ml) (all GIBCO BRL, Gent). After centrifugation, cells were loaded for 30 minutes with the Ca²⁺ sensitive fluorescent dye Fluo-3 AM (Molecular Probes, Leiden, Netherlands) (2 million cells/ml in RPMI medium containing 4 μM Fluo-3 AM, 20 mM HEPES, 0.1% Bovine Serum Albumin (BSA) and 5 mM probenecid). Excess dye was removed by 3-fold washing with buffer (5 mM HEPES, 140 mM NaCl, 1 mM MgCl₂, 5 mN KCl, 10 mM glucose, 2.5 mM probenecid, 1.25 mM CaCl₂, 0.1% BSA; all further incubations were done in this buffer). Cells were plated at a density of 150 000 cells/well in dark-wall 96-well plates (Costar, Cambridge, Mass.) and sedimented by centrifugation (1 minute). The cells were pre-incubated for 20 minutes with test compound Then, 10⁻⁷ M hMCP-1 (Bachem, Bubendorf, Switserland) was added. Changes in intracellular free Ca²⁺ concentration were measured using the Fluorescent Imaging Plate Reader (FLIPR, Molecular Devices, Munchen, Germany). Fluorescence was recorded every second from 10 seconds before the addition of the MCP-1 till 2 minutes after the addition (first minute: 60 records with 1 second intervals, second minute 20 records with 3 second intervals). The maximal fluorescence obtained during this time frame was used for further calculations.

Table 4 reports pIC₅₀ values obtained in the above-described test for compounds of formula (I). pIC₅₀ defines −log IC₅o wherein IC₅₀ is the molar concentration of the test compound which inhibits 50% of specific MCP-1 induced Ca²⁺ flux.

TABLE 4

| Comp. No. | pIC₅₀ |
|---|---|
| 5 | 6.7 |
| 26 | 6.66 |
| 14 | 6.38 |
| 11 | 6.14 |
| 1 | 7.45 |
| 12 | 6.4 |
| 20 | 7.38 |
| 19 | 6.08 |
| 24 | 6.2 |
| 21 | 7.46 |
| 9 | 7.48 |

Radioligand Binding Assay.

¹²⁵I-MCP binding assays performed in 96-well plates with 40 μg of protein per well. Compounds were dissolved and diluted in DMSO to 100× dilutions. A 10× concentration range of compounds was prepared in binding buffer (10% DSMO). Competition binding assay contained the following components in a total volume of 250 μl of the appropriate compound dilution (final concentration of 1% DMSO), 200 μl membranes from CCR2B-transfected CHO cells dissolved in binding buffer and 25 μl ¹²⁵I-MCP-1(Bolton and Hunter labeled, Amersham, specific activity=2000 Ci/mmol, 0.15 nM final). Binding buffer was composed of 25 mM HEPES, 5 mM MgCl₂, 1 mM CaCl₂, 0.5% protease-free bovine serum albumin, pH 7.4. After 90 minutes incubation at 25° C., membranes were harvested on GF/B filters -presoaked in 0.5% polyethylenimene, followeded by washing with buffer containing 25 mM HEPES, 5 mM MgCl₂, 1 mM CaCl₂, 5 M NaCl, pH 7.4. Filter bound radioactivity was determined by liquid scintillation counting. EC₅₀ values (μM) and Kᵢ values (μM) were calculated. The EC₅₀ value indicates the concentration of the test compound that competes with MCP-1 for half of the specific binding sites; the Kᵢ value indicates the equilibrium dissociation constant, i.e. the concentration of the test compound that will bind to half of the binding sites at equilibrium in the absence of radioligand or other competitors. EC₅₀ values and Kᵢ values were calculated using non-linear regression in Graphpad Prism. Prism calculates the Kᵢ or affinity of the receptor for the competing drug using the equation of Cheng and Prusoff (Biochem. Pharmacol. 1973, 22: 3099-3108). A low Kᵢ indicates a high affinity of the receptor for the test compound.

$${K}_{i} = \frac{{EC}_{50}}{1 + \frac{[ radioligand ]}{{K}_{d}}}$$

wherein K_{d} describes the affinity of the radioligand for the receptor, i.e. the concentration of the radioligand that will bind to half of the binding sites at equilibrium in the absence of competitors.

Compound 1 has a Kᵢ (μM) of 0.17.

Chemotactic Response

The CCR₂ antagonistic activity of the compounds of the present invention can also be determined by measuring the effect of the compounds on the chemotactic response of cells in the presence of a chemokine, such as for example MCP-1.

Mononuclear cells from human heparinized peripheral blood (PBMC) were isolated using Ficoll-Paque gradient centrifugation (Amersham Biosciences). Assays of chemotactic responsiveness were performed using disposable 96-well chemotaxis chambers (ChemoTx, Neuro Probe) with 5-μm pore size polycarbonate (PVP-free) filter membranes. Mononuclear cells were fluorescently labeled with 5 μg/ml Calcein-AM (Molecular Probes, Eugene, Oreg.) at 37° C. for 30 minutes. Labeled cells were washed twice and resuspended at 5×10⁶ cells/ml in Hanks' Balanced Salt Solution (Gibco BRL) supplemented with 0.2% bovine serum albumin. Subsequently, cells were pre-incubated for 10 minutes at room temperature with serial dilutions of the compounds in DMSO (dimethylsulfoxide) (final DMSO concentration of 0.2%). Bottom wells of the chemotaxis chamber were loaded with 28 μl medium containing 30 ng/ml recombinant hMCP-1 (R&D) or buffer only. Pre-treated cells (100.000 cells) were added in triplicate to the topside of the filter (20 μl ) and incubated at 37° C. in humidified air containing 5% CO₂. After 105 minutes incubation, the non-migrated cells were removed from the top of the filter by gently wiping the filter with a tissue. The migrated cells were measured using a fluorescent plate reader (λ_{excition}=485 nm; λₑₘᵢₛₛᵢₒₙ=538 nm). The chemotactic response can be expressed as chemotactic index (C.I.), being the ratio of the means of migrated cells in the presence of MCP-1 and the means of migrated cells in the absence of chemokine. Percentage inhibition can be calculated using the formula:

$$\% inhibition = ( 1 - \frac{{F}_{sample} - {F}_{buffer}}{{F}_{MCP \text{-} 1} - {F}_{buffer}} ) \times 100$$

with Fₛₐₘₚₗₑ, the fluorescence of the cells pre-incubated with 10, 1, 0.1, 0.01 or 0.001 μM compound and migrated to 30 ng/ml MCP-1 in the bottom wells; F_{MCP-1}, the fluorescence of the cells pre-incubated with buffer-0.2% DMSO and migrated to 30 ng/ml MCP-1 and F_{buffer}, the fluorescence of cells pre-incubated with buffer-0.2% DMSO and spontaneous migrated to buffer in the bottom wells.

1. A compound of formula (I) 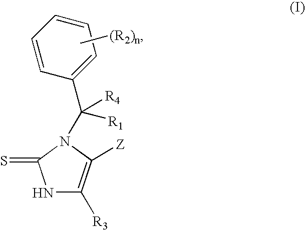
a N-oxide, a pharmaceutically acceptable addition salt, a quaternary amine or a stereochemically isomeric form thereof, wherein
R₁ represents hydrogen, C₁₋₆alkyl, C₃₋₇cycloalkyl, C₁₋₆alkyloxyC₁₋₆alkyl, di(C₁₋₆alkyl)aminoC₁₋₆alkyl, aryl or heteroaryl;
each R₂ independently represents halo, C₁₋₆alkyl, C₁₋₆alkyloxy, C₁₋₆alkylthio, polyhaloC₁₋₆alkyl, polyhaloC₁₋₆alkyloxy, cyano, aminocarbonyl, amino, mono- or di(C₁₋₄alkyl)amino, nitro, aryl or aryloxy;
R₃ represents hydrogen, cyano, C₁₋₆alkyl optionally substituted with hydroxy or C₁₋₆alkyloxy, C(═O)—O—R₅, C(═O)—NR₆ₐR_{6b}, C(═S)—NR₆ₐR_{6b}, S(═O)₂—NR₆ₐR_{6b} or C(═O)—R₇;
R₄ represents hydrogen or C₁₋₆alkyl;
R₅ represents hydrogen, C₁₋₆alkyl, hydroxyC₁₋₆alkyl, C₂₋₆alkenyl, C₂₋₆alkynyl, polyhaloC₁₋₆alkyl, C₁₋₆alkyloxyC₁₋₆alkyl, aminoC₁₋₆alkyl, mono- or di(C₁₋₄alkyl)aminoC₁₋₆alkyl, aminocarbonylC₁₋₆alkyl, mono- or di(C₁₋₄alkyl)aminocarbonylC₁₋₆alkyl or aryl;
R₆ₐ and R_{6b} each independently represent hydrogen, C₁₋₆alkyl, amino, mono- or di(C₁₋₄alkyl)amino, arylNH—, aminoC₁₋₆alkyl, mono- or di(C₁₋₄alkyl)amino-C₁₋₆alkyl, C₁₋₆alkylcarbonylamino, aminocarbonylamino, C₁₋₆alkyloxy, carbonylamino or hydroxyC₁₋₆alkyl; or
R₆ₐ and R_{6b} taken together with the nitrogen to which they are attached form pyrrolidinyl, imidazolidinyl, pyrazolidinyl, piperidinyl, piperazinyl, morpholinyl, thiomorpholinyl or piperazinyl substituted with C₁₋₆alkyl;
R₇ represents hydrogen, C₁₋₆alkyl, hydroxyC₁₋₆alkyl, C₂₋₆alkenyl, C₂₋₆alkynyl, polyhaloC₁₋₆alkyl, C₁₋₆alkyloxyC₁₋₆alkyl, aminoC₁₋₆alkyl, mono- or di(C₁₋₄alkyl)aminoC₁₋₆alkyl, aminocarbonylC₁₋₆alkyl, mono- or di(C₁₋₄alkyl)aminocarbonylC₁₋₆alkyl, aryl or heteroaryl;
Z represents a cyclic ring system selected from 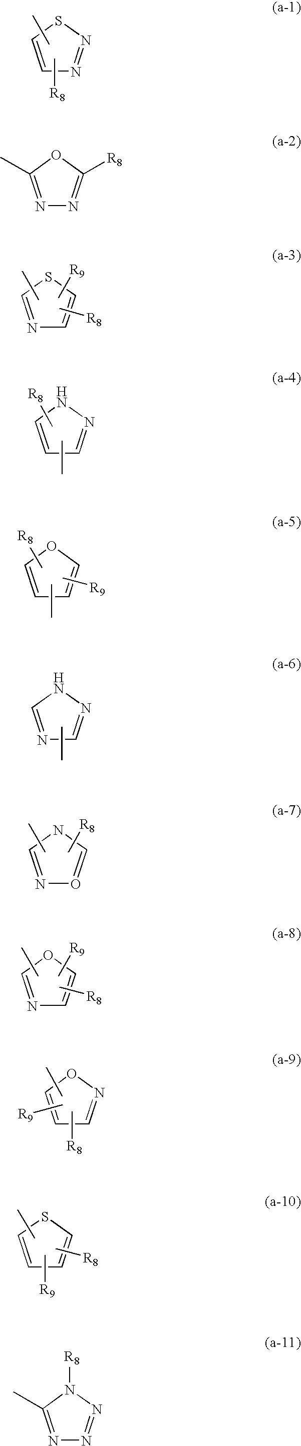 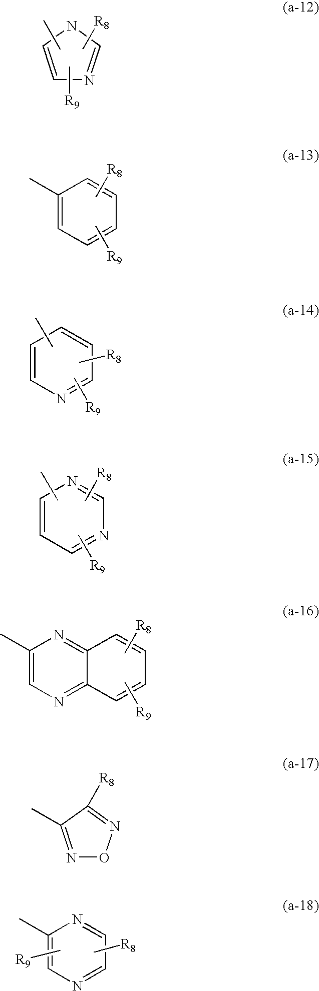
each R₈ independently represents hydrogen, halo, C₁₋₆alkyl, C₁₋₆alkyloxy, polyhaloC₁₋₆alkyl, polyhaloC₁₋₆alkyloxy, cyano, aminocarbonyl, mono- or di(C₁₋₄alkyl)aminocarbonyl, amino, mono- or di(C₁₋₄alkyl)amino, hydroxyC₁₋₆alkylamino, aryl, aryloxy, piperidinyl, piperidinylamino, morpholinyl, piperazinyl or nitro;
each R₉ independently represents hydrogen, halo or C₁₋₆alkyl;
n is 1, 2, 3, 4 or 5;
aryl represents phenyl or phenyl substituted with one, two, three, four or five substituents each independently selected from halo, C₁₋₆alkyl, C₁₋₆alkyloxy, polyhaloC₁₋₆alkyl, polyhaloC₁₋₆alkyloxy, cyano, aminocarbonyl, mono- or di(C₁₋₄alkyl)aminocarbonyl, amino, mono- or di(C₁₋₄alkyl)amino, phenyloxy or nitro;
heteroaryl represents furanyl, thienyl, pyrrolyl, oxazolyl, thiazolyl, imidazolyl, pyrazolyl, isoxazolyl, isothiazolyl, oxadiazolyl, triazolyl, thiadiazolyl, pyridyl, pyridazinyl, pyrimidinyl, pyrazinyl, each of said heterocycles optionally being substituted with one or two substituents each independently selected from halo, C₁₋₆alkyl, C₁₋₆alkyloxy, polyhaloC₁₋₆alkyl, polyhaloC₁₋₆alkyloxy, cyano, aminocarbonyl, mono- or di(C₁₋₄alkyl)aminocarbonyl, amino, mono- or di(C₁₋₄alkyl)amino or nitro.

2. A compound according to claim 1 wherein R₂ represents halo, polyhaloC₁₋₆alkyl or aryloxy.

3. A compound according to claim 2 wherein R₂ represents halo.

4. A compound according to claim 1 wherein Z represents a cyclic ring system selected from (a-2), (a-3), (a-4), (a-5), (a-6), (a-7), (a-11), (a-13), (a-14) or (a-15).

5. A compound according to claim 4 wherein Z represents a cyclic ring system selected from (a-2) or (a-15).

6. A compound according to claim 1 wherein R₃ represents hydrogen, cyano, C(═O)—O—R₅, C(═O)—NR₆ₐR_{6b}, C(═S)—NR₆ₐR_{6b}, S(═O)₂—NR₆ₐR_{6b} or C(═O)—R₇.

7. A compound according to claim 5 wherein R₃ represents hydrogen, C₁₋₆alkyl substituted with C₁₋₆alkyloxy or C(═O)—O—R₅.

8. A compound according to claim 7 wherein R₃ represents hydrogen or C(═O)—O—R₅.

9. A compound according to claim 1 wherein R₁ represents C₁₋₆alkyl.

10. A compound according to claim 1 wherein R₄ represents hydrogen.

11. A compound according to claim 1 wherein n is 2.

12. A compound according to claim 1 wherein R₁ represents C₁₋₆alkyl; R₂ represents halo, polyhaloC₁₋₆alkyl or aryloxy; R₃ represents hydrogen, C₁₋₆alkyl substituted with C₁₋₆alkyloxy, or C(═O)—O—R₅; Z represents a cyclic ring system selected from (a-2), (a-3), (a-4), (a-5), (a-6), (a-7), (a-11), (a-13), (a-14) or (a-15); R₄ represents hydrogen; n represents 1, 2 or 3.

13. A compound according to claim 1 wherein the compound is stereochemically pure.

14. (canceled)

15. A method for preventing or treating diseases mediated through activation of the CCR2 receptor comprising administering to a subject in need thereof a therapeutically effective amount of a compound as claimed in claim 1.

16. The method according to claim 15 wherein the disease is an inflammatory disease.

17. A pharmaceutical composition comprising a pharmaceutically acceptable carrier, and as active ingredient a therapeutically effective amount of a compound as claimed in claim 1.

18. A process of preparing a pharmaceutical composition comprising mixing a pharmaceutically acceptable carrier with a compound as claimed in claim 1.

19. A process of preparing a compound according to claim 1 comprising
a) reacting an intermediate of formula (II) with an appropriate acid optionally in the presence of a suitable solvent 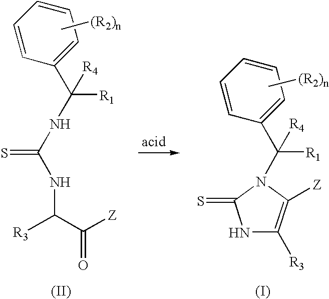
with R₁, R₂, R₃, R₄, Z and n as defined in claim 1;
b) reacting an intermediate of formula (III) with an intermediate of formula (IV) in the presence of a suitable solvent 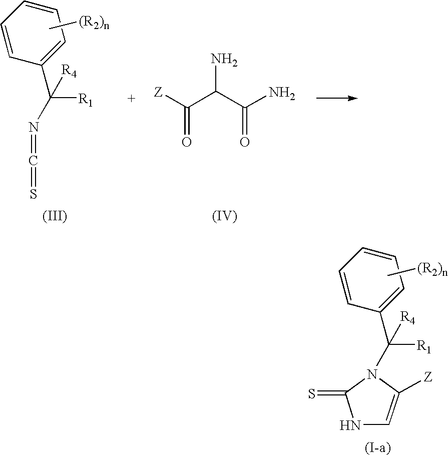
with R₁, R₂, R₄, Z and n as defined in claim 1;
c) reacting an intermediate of formula (V) with a suitable acid 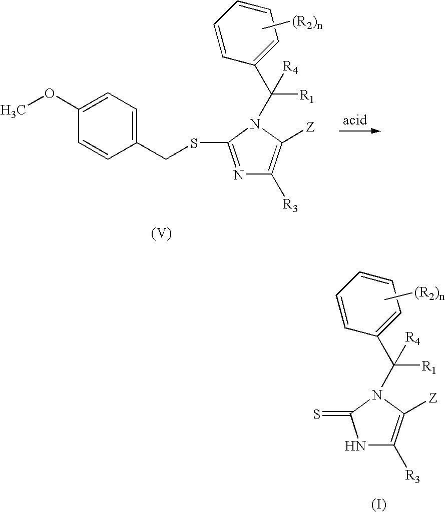
with R₁, R₂, R₃, R₄, Z and n as defined in claim 1;
d) reacting an intermediate of formula (VI) with phosphoric trichloride (POCl₃) or Burgess'reagent in the presence of a suitable solvent 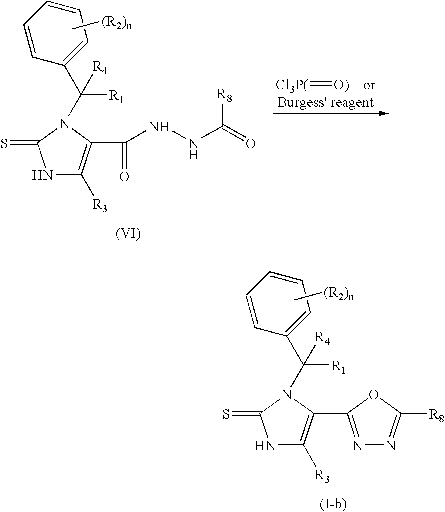
with R₁, R₂, R₃, R₄, R₈ and n as defined in claim 1;
e) reacting an intermediate of formula (VII) with SOCl₂ and HC(═O)NH—NH₂ in the presence of a suitable solvent 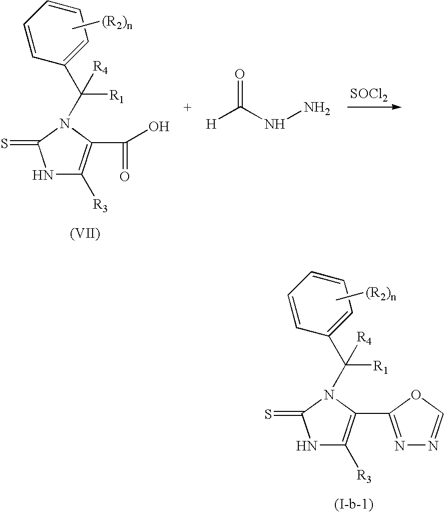
with R₁, R₂, R₃, R₄ and n as defined in claim 1;
f) reacting an intermediate of formula (VIII) with an intermediate of formula (IX) in the presence of a suitable base and a suitable solvent 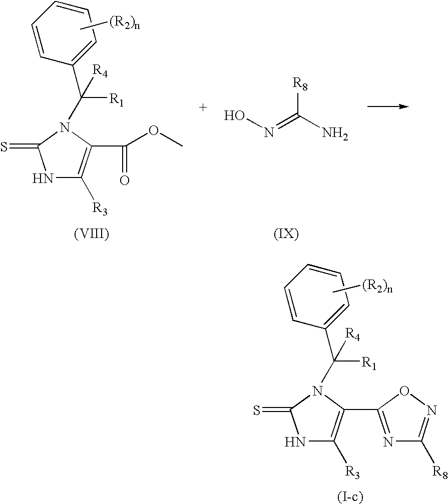
with R₁, R₂, R₃, R₄, R₈ and n as defined in claim 1;
g) reacting an intermediate of formula (X) with methyl formate, KSCN in the presence of a suitable base, a suitable acid and a suitable solvent, followed by reacting the thus obtained intermediate of formula (X-a) with Bu₃SnN₃ in the presence of a suitable solvent 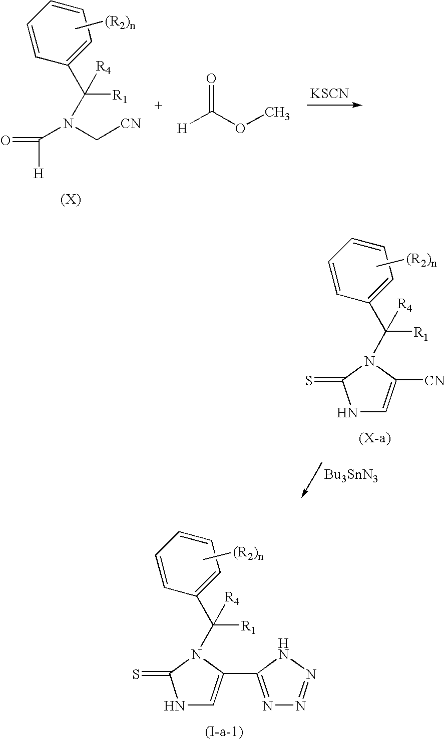
with R₁, R₂, R₄ and n as defined in claim 1;
or, if desired, converting compounds of formula (I) into each other following art-known transformations, and further, if desired, converting the compounds of formula (I), into a therapeutically active non-toxic acid addition salt by treatment with an acid, or into a therapeutically active non-toxic base addition salt by treatment with a base, or conversely, converting the acid addition salt form into the free base by treatment with alkali, or converting the base addition salt into the free acid by treatment with acid; and, if desired, preparing stereochemically isomeric forms, quaternary amines or N-oxide forms thereof.
